(19) United States
(12) Patent Application Publication
(10) Pub. No.: US 2016/0203262 A1
(43) Pub. Date: Jul. 14, 2016

(54) System and Method for Determining Quality of Stem Cell Derived Cardiac Myocytes

(71) Applicant: President and Fellows of Harvard College, Cambridge, MA (US)

(72) Inventors: Sean Paul Sheehy, Somerville, MA (US); Francesco Pasqualini, Milano (IT); Kevin Kit Parker, Waltham, MA (US)

(73) Assignee: President and Fellows of Harvard College, Cambridge, MA (US)

(21) Appl. No.: 14/913,925

(22) PCT Filed: Aug. 21, 2014
(86) PCT No.: PCT/US14/52125

§ 371 Date: Feb. 23, 2016

Related U.S. Application Data
(60) Provisional application No. 61/869,347, filed on Aug. 23, 2013.

(51) Int. Cl. G06F 19/24; G01N 33/50

(52) U.S. Cl. CPC G06F 19/24; G01N 33/5091

(57) ABSTRACT

The present invention relates to a system and method for calculating a quality index of a differentiated cell. To calculate the quality index, the present invention measures a differentiated cell by at least one metric, calculates a strictly standardized mean difference between the differentiated cell and a targeted cell, and calculates a mean squared error versus the target cell to define a value that represents the total difference between the differentiated cell and targeted cell based on the at least one measured metric.

### CROSS-REFERENCES TO RELATED APPLICATIONS

This application is the U.S. national stage application filed under 35 §U.S.C. 371 claiming benefit to International Patent Application No. PCT/US2014/052125, filed Aug. 21, 2014, which is entitled to priority under 35 U.S.C. §119(e) to U.S. Provisional Patent Application No. 61/869,347, filed Aug. 23, 2013, each of which applications are incorporated by reference herein in their entireties.

### STATEMENT REGARDING FEDERALLY SPONSORED RESEARCH OR DEVELOPMENT

This invention was made with government support under grant nos. U01 HL100408-02 and UH2 TR000522-01, awarded by the National Institutes of Health (NIH). The government has certain rights in the invention.

### BACKGROUND OF THE INVENTION

In response to widespread efforts to commercialize differentiated stem cells (Brower, 1999, Nat Biotechnol 17:139-142), the U.S. Food and Drug Administration (FDA) established a set of regulations and guidelines for manufacturing and quality control evaluation of human cellular and tissue-based products derived from stem cells (Current Good Tissue Practice (CGTP) and Additional Requirements for Manufacturers of Human Cells, Tissues, and Cellular and Tissue-Based Products (HCT/Ps). Food and Drug Administration Center for Biologics Evaluation and Research (2011)). The recommendations outlined for evaluating differentiated stem cell phenotype were developed specifically to address patient safety concerns, such as tumorigenicity and immunologic incompatibility due to the initial focus of the industry on regenerative medicine applications (Fink, 2009, Science 324:1662-1663). Concerns over patient safety may have slowed the commercialization of regenerative therapies (Fox, 2011, Nat Biotechnol 29:375-376), but the use of industrial stem cell-based products for in vitro research, particularly pharmaceutical screening applications (Rubin, 2008, Cell 132:549-552; Wobus and Loser, 2011, Arch Toxicol 85:79-117) is a promising goal that can potentially be reached in the near term.

Due to the mandate to test all drug compounds for potential adverse effects on the heart, in vitro cardiac toxicity screening is a particularly important application that has prompted the development of commercial stem cell-derived cardiac myocytes by a number of companies

(Webb, 2009, Nat Biotechnol 27:977-979). In this context, the focus of quality assurance shifts from patient safety concerns to the development and adoption of measures that ensure these cells reliably mimic cardiac myocytes found in vivo. Unfortunately, no standardized guidelines currently exist for the comprehensive evaluation of structure, function and gene expression profile in stem cell derived myocytes. As a result, it is unclear whether the various stem cell-derived myocyte cell lines on the market exhibit comparable performance to one another, or if any of them accurately recapitulate the characteristics of native myocytes.

Thus, there is a need in the art for a quality assessment routine that involves relevant measurement parameters that are representative of downstream phenotypic development from stem cells, such as the ventricular myocyte phenotype derived from stem cell lines. The present invention satisfies these needs.

### SUMMARY OF THE INVENTION

The present invention relates to a method for calculating a quality index of a differentiated cell. The method includes the steps of measuring a differentiated cell by at least one metric, calculating a normalized residue, such as a strictly standardized mean difference between the differentiated cell and a targeted cell, and calculating a mean squared error versus the target cell to define a value that represents the total difference between the differentiated cell and targeted cell based on the at least one measured metric. In one embodiment, the at least one metric is selected from the group consisting of genetic information, electrophysiological information, structural information, and contractile information. In another embodiment, the at least one metric includes each of genetic information, electrophysiological information, structural information, and contractile information. In another embodiment, the differentiated cell is derived from a potent cell. In another embodiment, the potent cell is a stem cell. In another embodiment, the differentiated cell is a myocyte. In another embodiment, the at least one metric is a sarcomere packing density. In another embodiment, information pertaining to the targeted cell is a predetermined value related to the at least one metric. In another embodiment, a lower MSE value is indicative of greater similarity between the differentiated cell and the targeted cell.

The present invention also relates to a systsem for calculating a quality index of a differentiated cell. The system includes a software platform run on a computing device that calculates a normalized residue, such as a strictly standardized mean difference between a differentiated cell and a targeted cell, and calculates a mean squared error versus the target cell to define a value that represents the total difference between the differentiated cell and targeted cell based on at least one measured metric of the differentiated cell.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A-1E, is a comparison of mES, miPS and neonate gene expression profiles on isotropic and anisotropic ECM substrates. FIG. 1A depicts culturing (i) mES, (ii) miPS and (iii) neonate myocytes on substrates with a uniform coating of FN resulted in isotropic cellular arrangement. FIG. 1B are volcano plots showing statistical comparisons of qPCR measurements of cardiac genes between (i) mES and neonate isotropic monolayers, and between (ii) miPS and neonate isotropic monolayers that reveal significant differences for a number of genes (two-tailed T-test, n=3 for all conditions, points on the plot colored green or red represent genes with p<0.05). FIG. 1C depicts culturing (i) mES, (ii) miPS and (iii) neonate myocytes on substrates with micro-contact printed lines of FN that were 20 μm wide and spaced 4 μm apart resulted in anisotropic cellular arrangement in all three cell types. FIG. 1D are volcano plots showing statistical comparisons of qPCR measurements of cardiac genes between (i) mES and neonate anisotropic monolayers, and between (ii) miPS and neonate anisotropic monolayers that reveal slightly fewer genes demonstrating significant differences than in the isotropic cultures (two-tailed T-test, n=3 for all conditions, points on the plot colored green or red represent genes with p<0.05). FIG. 1E depicts hierarchical clustering of mean 2^{-ΔCt} values for a select panel of genes representing the components of the sarcomere, ion channel subunits, and genes commonly used to deduce ventricular vs. atrial identity that revealed a distinct separation between the neonate expression profile and the expression profiles of the mES and miPS engineered tissues. Scale bars=100 μm.

FIG. 2, comprising FIGS. 2A-2E, is a comparison of myofibril architecture in mES, miPS and neonate engineered tissues. Immunofluorescence visualization of sarcomeric α-actinin in FIG. 2A depict isotropic monolayers of (i) mES, (ii) miPS, and (iii) neonate myocytes and in FIG. 2B depict anisotropic monolayers of (i) mES, (ii) miPS, and (iii) neonate myocytes revealed the pattern of sarcomere organization adopted by each cell type in response to geometric cues encoded in the ECM. Immature premyofibrils (red arrows) were observed exclusively in mES and miPS engineered tissues. Quantitative evaluation of sarcomeric α-actinin immunofluorescence micrographs allowed statistical comparison of sarcomere organization and architecture. As shown in FIG. 2C, orientational order parameter (OOP) was used as a metric of global sarcomere alignment within the engineered tissues and showed that anisotropic neonate tissues exhibited significantly greater overall sarcomere alignment than the mES and miPS anisotropic tissues. No significant differences in global sarcomere alignment were observed between the isotropic mES, miPS and neonate tissues. FIG. 2D is a comparison of z-line spacing that revealed the neonate anisotropic tissues exhibited significantly greater sarcomere length than both the mES and miPS anisotropic tissues. As shown in FIG. 2E, from the measurements of sarcomere length, sarcomere packing density was calculated for anisotropic tissues of each cell type. All three cell types exhibited significantly different sarcomere packing densities from the two other cell types, indicating that each type of myocyte gave rise to a unique sarcomere packing density. All results presented as mean±standard error of the mean. Statistical tests used were either ANOVA (*=p<0.05), or ANOVA on ranks (†=p<0.05). Scale bars=10 μm.

FIG. 3, comprising FIGS. 3A-3H, is a comparison of electrical activity in mES, miPS and neonate engineered tissues. As depicted in FIG. 3A, patch clamp recordings taken on isolated mES, miPS and neonate myocytes exhibited action potentials (AP) with both (i) ventricular-like, and (ii) atrial-like profiles. As depicted in FIG. 3B, characterization of the AP traces revealed no significant differences between the three cell types, but the mES and miPS myocytes exhibited an equal proportion of ventricular-like (mES-v, miPS-v) and atrial-like (mES-a, miPS-a) AP traces, whereas the neonates exhibited primarily ventricular-like (neonate-v) AP profiles. As depicted in FIG. 3C the electrophysiological characteristics of anisotropic (i) mES, (ii) miPS, and (iii) neonate tissues were assessed using optical mapping and the photovoltaic dye RH237. As depicted in FIG. 3D, comparison of conduction properties between the mES, miPS and neonate tissues revealed no significant differences in either longitudinal (LCV) or transverse (TCV) conduction velocity. As depicted in FIG. 3E, evaluation of optical AP duration in anisotropic tissues revealed no significant differences in APD50, but a significant difference in APD90 between mES and neonate tissues was observed. As depicted in FIG. 3F, comparison of Ca²⁺ transients measured in anisotropic tissues revealed that the 50% decay time (CaT50) of the miPS tissues was significantly lower than the both the mES and neonate tissues, but the 90% decay time (CaT90) of both the mES and miPS tissues was significantly lower than the neonate tissues. As depicted in FIG. 3G, patch clamp recordings were collected on isolated mES, miPS, and neonate myocytes to measure and compare (i) L-type, and (ii) T-type Ca²⁺ current densities elicited at various holding potentials. As depicted in FIG. 3H, patch clamp recordings of maximum Ca²⁺ current density in isolated mES, miPS and neonate myocytes revealed a significant difference in total Ca²⁺ current density (TOT) between the neonate and mES myocytes. No significant differences in L-type Ca²⁺ current density (LCC) were observed, but a significant difference in T-type Ca²⁺ current density (TCC) was observed between the neonate and mES myocytes. All results presented as mean±standard error of the mean. Statistical test used was ANOVA (*=p<0.05). Scale bars=20 μm.

FIG. 4, comprising FIGS. 4A-4D depicts the comparison of contractile performance in mES, miPS, and neonate engineered tissues. As shown in FIG. 4A, contractile performance of anisotropic mES, miPS, and neonate tissues was assessed using the muscular thin film (MTF) assay wherein the radius of curvature of the MTFs at (i) diastole and (ii) peak systole were used to calculate contractile stress. As depicted in FIG. 4B, the radius of curvature of the MTFs was used to calculate and compare the temporal contractile strength profiles of anisotropic mES (green), miPS (red), and neonate (blue) tissues. As depicted in FIG. 4C, comparison of MTF contractile output revealed that neonate anisotropic tissues generated significantly greater diastolic, peak systolic, and twitch stress than both the mES and miPS tissues. Depicted in FIG. 4D, is a graphical representation of action potential morphology (black solid line), calcium transient morphology (blue dotted line), and contractility profile (red dotted line) during a typical excitation-contraction cycle of the mES, miPS, and neonate engineered anisotropic tissues. Statistical test used was ANOVA (*=p<0.05).

FIG. 5 is an integrated visual comparison of mES, miPS, and neonate experimental measurements. Strictly Standardized Mean Difference (β) values were computed for mES- and miPS-derived myocytes relative to the neonate cardiac myocytes from the mean and sample standard deviations collected for each experimental measurement. Descriptions for each abbreviation listed in the right-hand column can be found in Table 2. These β values were organized by measurement type (i.e. gene expression, myocyte architecture, electrophysiology, contractility) and plotted to allow comparison. Negative β values indicate measurements with higher relative magnitude in the neonate cardiac myocytes, whereas positive β values indicate measurements that were higher in the mES/miPS myocytes relative to the neonate cardiac myocytes.

FIG. 6, comprising FIGS. 6A-6F, is an evaluation of myocyte morphology. FIG. 6A depicts isotropic cultures of (i) mES, (ii) miPS, and (iii) neonate cardiac myocytes fixed and immunostained for the presence of sarcomeric α-actinin (red), F-actin (green), and chromatin (blue). Cardiac myocytes were identified by the presence of sarcomeric α-actinin positive z-lines, and the boundaries of fully spread, mono-nucleated myocytes were manually traced using the polygon tool in ImageJ. The total number of pixels contained within each traced polygon was used to calculate cellular aspect ratio (FIG. 6B), and the total spread surface area (FIG. 6C) for each cell type. Similarly, the voltage sensitive dye RH237 used for optical mapping experiments allowed identification of myocyte boundaries in anisotropic monolayers of mES (FIG. 6D(i)), miPS (FIG. 6D(ii)), and neonate cardiac myocytes (FIG. 6D(iii)). The total number of pixels contained in each manually traced outline was used to calculate aspect ratio (FIG. 6E), and total spread surface area (FIG. 6F) for each type of myocyte. All results presented as mean±standard error of the mean. Statistical tests used was ANOVA on ranks (†=p<0.05). Scale bars=20 μm.

FIG. 7, comprising FIGS. 7A-7C, depicts sarcomere structural characterization. Image processing flow in FIG. 7A: sarcomeric α-actinin immunographs were deconvolved, projected onto a single 2D image and then processed with a tubeness operator before further processing. In FIG. 7B, the orientations of sarcomeric α-actinin positive pixels were detected with a structure tensor method, color coded using the hsv digital image representation (FIG. 7B(i)) and finally displayed into a histogram (FIG. 7B(ii)) of the normalized occurrences of each orientation. In FIG. 7C, the sarcomere length and the overall regularity of the cytoskeletal structure were detected processing the immunograph 2D Fast Fourier Transform algorithm. The detected power spectrum (FIG. 7C(i)), (for representation purpose a gamma correction of 0.1 was applied) was then integrated and normalized by the total energy. In FIG. 7C(ii), the sarcomere packing density was defined as the area under the signal peaks (red curve) whose location related with the sarcomere length.

FIG. 8, comprising FIGS. 8A-8D, depict ratiometric Ca²⁺ transient measurements. In FIG. 8A, anisotropic tissues were loaded with Fura-red and 20 lines (white box, direction indicated by the white arrow) were scanned in dual-excitation mode at 405 nm (FIG. 8A(i)) and 488 nm (FIG. 8A(ii)); the sampling frequency was 250 Hz. Scale bars=15 In FIG. 8B, the background-subtracted averaged number of photons collected with excitation at 405 nm (blue) and 488 nm (green) in each frame was used to obtain 2 signals proportional to the elevation of the cytoplasmic calcium in the tissue. In FIG. 8C, the ratio of these signals is an improved measurement of the calcium transient as bleaching and other artifacts are automatically corrected for. To further improve signal quality, 4-6 steady-state transients (grey box) were averaged (FIG. 8D) and the following quantities were calculated: diastolic level (grey box), peak level (*), time to peak (T2P) and the duration of the calcium transient at 50% (CaT50) and 90% (CaT90) decay.

FIG. 9, comprising FIGS. 9A-9E, show representative cardiomyocytes from neonate mouse (FIG. 9A, pCM), and mouse (FIG. 9B) or human (FIG. 9C) induced pluripotent stem cells (respectively miCM and hiCM) derived cardiomyocytes on square fibronectin islands. FIGS. 9(A-C)(i) show overlays of α-actinin (gray) and chromatin (blue) representative immunographs. FIGS. 9A-C(ii) show Hue Saturation Value (hsv) representation of the α-actinin channels in FIGS. 9A-C(iii): the Hue channel was used to color-code each sarcomeric α-actinin positive pixel with its detected orientation. FIGS. 9A-C(iii) show 2D Fourier power spectra corresponding to micrographs in FIGS. 9A-C(i) (for representation purposes only the images have a γ-correction of 0.1). FIG. 9D is a 1D representation of the 2D power spectrum in FIG. 9A(ii) (blue curve) and non-linear fitting with periodic (red curve) and aperiodic (black curve) components. The sarcomeric packing density is obtained from the area under the periodic component (red shaded area). FIG. 9E is a quantitative analysis of cytoskeletal organization with nuclear eccentricity E, orientational order parameter (OOP) and sarcomeric packing density (ε). Data are represented as mean±standard error of the means, n=3 for each condition.

FIG. 10, comprising FIGS. 10A-10C, is a schematic representation of myofibrillogenesis. FIG. 10A depicts the actin (green) cytoskeleton self-assembles during cell spreading; sarcomeric α-actinin (red) is initially diffuse in the cytoplasm. As shown in FIG. 10B, during cytoskeleton maturation, sarcomeric α-actinin localizes along the actin bundles in puncta known as Z-bodies, either at discrete locations or in relatively long stretches. As shown in FIG. 10C, when myofibrillogenesis is complete, sarcomeric α-actinin is localized in a regular lattice composed by Z-disks, ultra-structural units that signal the extremities of the sarcomere. The nuclear chromatin is indicated in blue.

FIG. 11, comprising FIG. 11A and FIG. 11B depicts migratory fibroblast (FIG. 11A) patterned on square fibronectin islands (FIG. 11B) exhibited an actin cytoskeletal structure characterized by the presence cortical actin at the cell borders and ring-like actin stress fibers in the perinuclear region. Actin (green), vimentin (red), chromatin (blue). Scalebar: 15 FIG. 12 depicts intrinsic cytoskeletal bias in primary cardiomyocytes (i) and murine (ii) or human (iii) induced pluripotent stem cell derived cardiomyocytes. When cultured on substrates uniformly coated with fibronectin, cardiomyocytes assume a pleomorphic shape, sustained by a cytoskeletal architecture that is the sole expression of the cell intrinsic bias, as there are no engineered boundary conditions. Under those circumstances, mononucleated (chromatin signal is encoded in blue channel) pCMs (i) and miCMs (ii) showed polarized myofibrils exhibited periodic striation of actin (green) and sarcomeric α-actinin (red). On the contrary hiCMs (iii) showed diffuse cortical actin and ring-like myofibrils. Scale bar: 15 μm.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical platforms for assessing quality of biological cell lines. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

As used herein, a “potent” cell refers to any cell that is capable of at least some differentiation. Also as used herein, a “differentiated cell” refers to any cell that has at least partially differentiated from a potent cell. Further, a “target cell” refers to the cell that the differentiated cell is being compared to, in determination of how closely the differentiated cell resembles the target cell according to at least one measurable metric.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### DESCRIPTION

The present invention includes a system and method for quality assessment of stem cell derived cells, cell populations and tissues. In one embodiment, the stem cell-derived cells are at least partially differentiated cells. In another embodiment, the stem cell-derived cells are specialized cells. The present invention allows a user to identify differences in one or more properties of differentiated cell tissues versus the target cell tissues that have important implications for their utility. The present invention also allows users to identify the commercial differentiated cell product lines that are most suitable for their needs, and of the underlying potent cells producing these differentiated cells. The present invention allows users to focus their efforts to improve cell differentiation protocols, and further serves as a robust quality control procedure for ensuring that batches of potent cells reach the desired differentiated cell phenotype.

### Method for Calculating a Quality Index

As contemplated herein, quality assessment is made by calculating a quality index based on at least one measurable metric which may include, without limitation, factors pertaining to one or more of genetic, electrophysiological, structural, and contractile information expressed as a numerical value. In some embodiments, the metric may relate to cytoskeletal organization, such as the sarcomere packing density of cardiomyocytes. It should be appreciated that the system and method of the present invention is not limited to these particular metrics, but instead may include any measurable metric of a cell or cell phenotype, provided such metric can be expressed as a value or score. Further, the quality index may be calculated based on just one metric, or it may be calculated based on a plurality of metrics. As contemplated herein, any combination of metrics may be used, the number and type of metrics being used generally depending on the type of differentiated cells being evaluated, or any other factors as determined by the user of the present invention.

These measurements are further made against a target cell, or alternatively against pre-calculated values for a target cell, such as a set of standard values to a target cell type. For example, the system and method of the present invention can assess the quality of stem cell derived myocytes, based on the integration of genetic, electrophysiological, structural, and contractile measurements, coupled with comparison against values for these measurements that are representative of the ventricular myocyte phenotype. In this embodiment, the efficacy of this procedure can be evaluated using commercially-available murine ES- (mES) and iPS- (miPS) derived myocytes compared against neonatal mouse ventricular myocytes (neonate).

While the present invention is focused primarily on stem cell-derived myocytes, it should be appreciated that the present invention is not limited to a particular cell type. Rather, the present invention allows for the calculation of a quality index for any type of biological cell, population of cells or tissue, as derived from any type of cell having the ability to differentiate, such as a stem cell, a progenitor and the like.

To determine how closely the differentiated cells match the phenotype of the target cells (or predetermined target cell values), the present invention integrates at least one measured metric of the differentiated cells, and calculates the difference, referred to herein as the “normalized residue,” between the at least one measured metric of the differentiated cells against the target cells or predetermined target cell values. For example, in one embodiment, for each experimental measurement, these values may be normalized, such as to the interval [0,1] and calculated the strictly standardized mean difference (denoted herein as β) according to the following:

$$β = \frac{{μ}_{1} - {μ}_{2}}{\sqrt{{σ}_{1}^{2} + {σ}_{2}^{2}}}$$

where μ represents mean and σ represents standard deviation, to evaluate the magnitude of difference, taking into account the variance in the measurements, between the differentiated cells and the target cells. This allows for determination of the effect size for each experimental measurement and for identification of the parameters that show the greatest degree of similarity and difference from the target cell tissues. The normalized residues, or β values, may be used from each experimental measurement to calculate the mean squared error (MSE) versus the target cell tissues according to the following:

$$MSE = \frac{1}{n} {\sum }_{i = 1}^{n} {β}_{i}^{2}$$

to define a single value that represents the total difference between the differentiated cells and target cells based on the measurements performed.

Accordingly, the MSE may be used herein as a quality index to provide a numeric score of how closely the differentiated cells match one or more characteristics of the target cells. The combination of measurable metrics employed allows a user of the system and method of the present invention to pin-point specific differences in one or more properties of engineered differentiated cell tissues versus the target cell tissues that have important implications for their utility in in vitro assays of tissue function. Further, this “quality index” not only allows users to identify the commercial differentiated cell product lines that are most suitable for their needs, it also provides insight to the source of the underlying potent cells producing these differentiated cells. The system and method of the present invention allows users may better understand where to focus their research and development efforts to improve their differentiation protocols, and further serves as a robust quality control procedure for ensuring that batches of potent cells released to customers faithfully recapitulate the desired differentiated cell phenotype.

As contemplated herein, calculation of the MSE may include a mechanism by which to weight each information item or measurable component for any metric, and to calculate a value that is determinative of that metric. In one embodiment, the lower the MSE value, the closer the differentiated cells are to the target cells. It should be appreciated that the values designated for each information item may vary according to the metric being measured. Further, the number or combination of information item categories will also effect the values designated. Depending on the application, one or more MSE scores may be set as a threshold value, where a score of equal to or above a designated value is indicative or predictive of quality. Alternatively, final score ranges can be used to designate categories of quality. It should be appreciated that the system of the present invention is not limited to any predetermined value, number, scale or other nomenclature for the MSE.

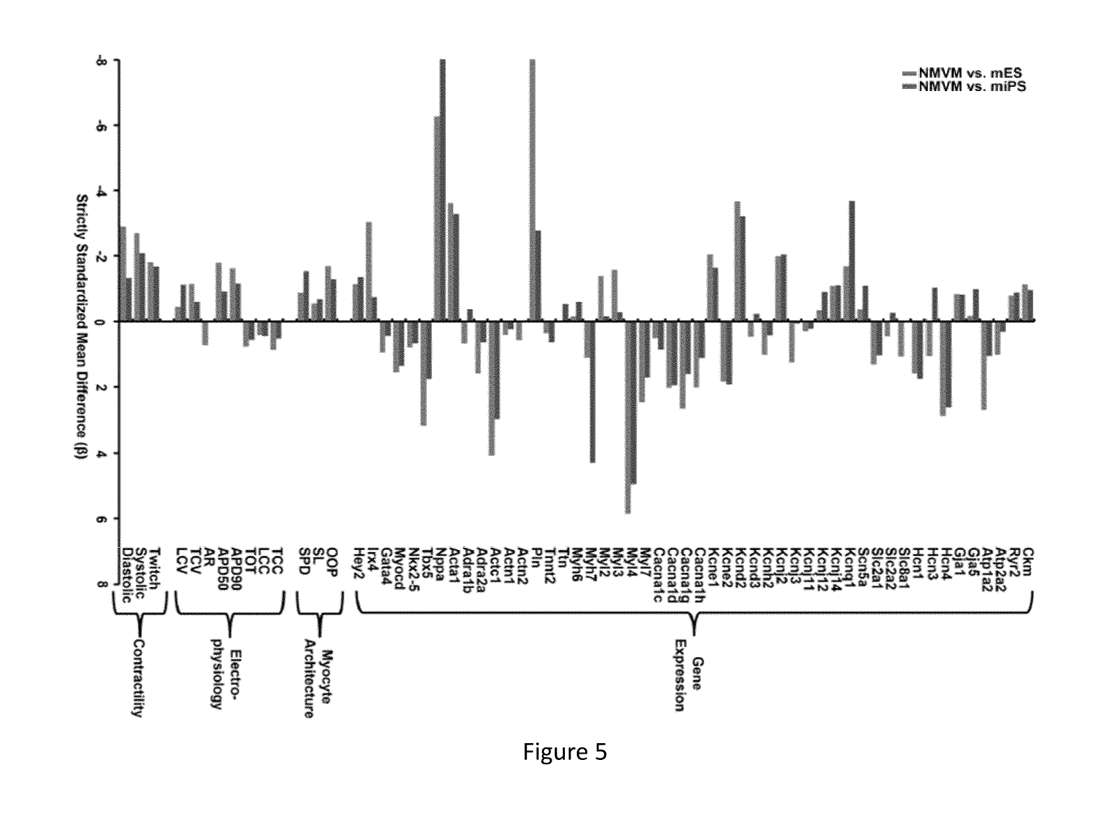

For example, as described in Example 1 herein, the β values presented in FIG. 5 resulted in an MSE score of 4.95 between the mES (differentiated cells) and neonate cardiac myocytes (target cells), whereas the miPS myocytes (differentiated cells) resulted in an MSE score of 3.60 from the neonate cardiac myocytes (target cells). In this example, the miPS myocytes exhibited a global phenotype that was slightly closer to the neonate cardiac myocytes than the mES-derived myocytes, although both the mES and miPS myocytes demonstrated substantial differences from the neonate cardiac myocytes for a number of characteristics.

### System Platform

As contemplated herein, the present invention includes a system platform for performing the aforementioned methods for quality assessment of differentiated cells derived from potent cells. In some embodiments, the system of the present invention may operate on a computer platform, such as a local or remote executable software platform, or as a hosted internet or network program or portal. In certain embodiments, only portions of the system may be computer operated, or in other embodiments, the entire system may be computer operated. As contemplated herein, any computing device as would be understood by those skilled in the art may be used with the system, including desktop or mobile devices, laptops, desktops, tablets, smartphones or other wireless digital/cellular phones, televisions or other thin client devices as would be understood by those skilled in the art. The platform is fully integratable for use with any additional platform and data output that may be used, for example with the measurement of a particular metric.

For example, the computer operable component(s) of the system may reside entirely on a single computing device, or may reside on a central server and run on any number of end-user devices via a communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data formats between two or more computing devices. There are no limitations to the number, type or connectivity of the databases utilized by the system of the present invention. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

Further, the communications network may also use standard architecture and protocols as understood by those skilled in the art, such as, for example, a packet switched network for transporting information and packets in accordance with a standard transmission control protocol/Internet protocol (“TCP/IP”). Any of the computing devices may be communicatively connected into the communications network through, for example, a traditional telephone service connection using a conventional modem, an integrated services digital network (“ISDN”), a cable connection including a data over cable system interface specification (“DOCSIS”) cable modem, a digital subscriber line (“DSL”), a T1 line, or any other mechanism as understood by those skilled in the art. Additionally, the system may utilize any conventional operating platform or combination of platforms (Windows, Mac OS, Unix, Linux, Android, etc.) and may utilize any conventional networking and communications software as would be understood by those skilled in the art.

To protect data, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. For example, a system administrator may allow for administration at one or more levels, such as at an individual reviewer, a review team manager, a quality control review manager, or a system manager. A system administrator may also implement access or use restrictions for users at any level. Such restrictions may include, for example, the assignment of user names and passwords that allow the use of the present invention, or the selection of one or more data types that the subservient user is allowed to view or manipulate.

As mentioned previously, the system may operate as application software, which may be managed by a local or remote computing device. The software may include a software framework or architecture that optimizes ease of use of at least one existing software platform, and that may also extend the capabilities of at least one existing software platform. The application architecture may approximate the actual way users organize and manage electronic files, and thus may organize use activities in a natural, coherent manner while delivering use activities through a simple, consistent, and intuitive interface within each application and across applications. The architecture may also be reusable, providing plug-in capability to any number of applications, without extensive re-programming, which may enable parties outside of the system to create components that plug into the architecture. Thus, software or portals in the architecture may be extensible and new software or portals may be created for the architecture by any party.

The system may provide software applications accessible to one or more users to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system.

The system software may also be a portal or SaaS that provides, via the GUI, remote access to and from the system of the present invention. The software may include, for example, a network browser, as well as other standard applications. The software may also include the ability, either automatically based upon a user request in another application, or by a user request, to search, or otherwise retrieve particular data from one or more remote points, such as on the internet or from a limited or restricted database. The software may vary by user type, or may be available to only a certain user type, depending on the needs of the system. Users may have some portions, or all of the application software resident on a local computing device, or may simply have linking mechanisms, as understood by those skilled in the art, to link a computing device to the software running on a central server via the communications network, for example. As such, any device having, or having access to, the software may be capable of uploading, or downloading, any information item or data collection item, or informational files to be associated with such files.

Presentation of data through the software may be in any sort and number of selectable formats. For example, a multi-layer format may be used, wherein additional information is available by viewing successively lower layers of presented information. Such layers may be made available by the use of drop down menus, tabbed folder files, or other layering techniques understood by those skilled in the art or through a novel natural language interface as described hereinthroughout. Formats may also include AutoFill functionality, wherein data may be filled responsively to the entry of partial data in a particular field by the user. All formats may be in standard readable formats, such as XML. The software may further incorporate standard features typically found in applications, such as, for example, a front or “main” page to present a user with various selectable options for use or organization of information item collection fields.

The system software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text or phone call, to alert a manager, expert, researcher, or other professional of the particular results. Further embodiments of such mechanisms are described elsewhere herein or may standard systems understood by those skilled in the art.

Accordingly, the system of the present invention may be used for calculating a quality index of a differentiated cell. The system may include a software platform run on a computing device that calculates the normalized residue, such as a strictly standardized mean difference (β), between a differentiated cell and a targeted cell, and calculates a mean squared error (MSE) versus the target cell to define a value that represents the total difference between the differentiated cell and targeted cell based on at least one measured metric of the differentiated cell.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Assessment of Stem Cell Derived Myocyte Differentiation

In order to develop quality assurance standards for assessing stem cell-derived myocyte differentiation, it is necessary to first establish a set of characteristics that reliably define cardiac myocyte identity. In one example such characteristics may include evaluation of form and function that give rise to the contractile properties of cardiac myocytes in the healthy, post-natal heart. In addition to measuring the expression of cardiac biomarker genes (Ng et al., 2010, Am J Physiol Cell Physiol 299:C1234-1249; Bruneau, 2002, Circ Res 90:509-519), the organizational characteristics of the contractile myofibrils (Feinberg et al., 2012, Biomaterials 33:5732-5741), the electrical activity that regulates myofibril contraction (Kleber and Rudy, 2004, Physiol Rev 84:431-488), and the contractile force output of the myofibrils directly (Alford et al., 2010, Biomaterials 31:3613-3621) were also examined. Since human ventricular myocytes are not readily available, commercially-available murine ES- (mES) and iPS- (miPS) derived myocytes were used, and these were compared against ventricular myocytes isolated from neonatal mice. Accordingly, the following example demonstrates the utility of comparing stem cell-derived myocytes and isolated cardiac myocytes possessing the desired phenotype using a multi-factorial comparison of high level myocardial tissue architectural and functional characteristics.

The following materials and methods were used in Example 1.

### Stem Cell-Derived Myocyte Culture

CorAt murine ES- and iPS-derived myocytes were cultured according to instructions, and with culture reagents supplied by the manufacturer (Axiogenesis, Cologne, Germany). Briefly, cells were cultured in T25 flasks pre-coated with 10 mg/ml fibronectin (FN) (BD Biosciences, Bedford, Mass.) in puromycin-containing culture media at 37° C. and 5% CO₂ for 24 hours, and in media that does not contain puromycin thereafter. After 72 hours, cells were dissociated with 0.25% trypsin and seeded onto micro-contact printed substrates at densities of 100,000/cm². Cells were cultured for 2 days on micro-contact printed substrates prior to experimentation.

### Neonatal Mouse Ventricular Myocyte Culture

Neonatal mouse ventricular myocytes were isolated from 2-day old neonatal Balb/c mice using procedures approved by the Harvard University Animal Care and Use Committee. Briefly, excised ventricular tissue was incubated in a 0.1% (w/v) trypsin (USB Corp., Cleveland, Ohio) solution cooled to 4° C. for approximately 12 hours with agitation. Trypsinized ventricular tissue was dissociated into cellular constituents via serial exposure to a 0.1% (w/v) solution of collagenase type II (Worthington Biochemical, Lakewood, N.J.) at 37° C. for 2 minutes. Isolated myocytes were maintained in a culture medium consisting of Medium 199 (Invitrogen, Carlsbad, Calif.) supplemented with 10% (v/v) heat-inactivated fetal bovine serum (FBS), 10 mM HEPES, 20 mM glucose, 2 mM L-glutamine, 1.5 μL vitamin B-12, and 50 U/ml penicillin and seeded at a density of 200,000 cells/cm². From the second day of culture onward, the FBS concentration was reduced to 2% (v/v), and medium was exchanged every 48 hours. Myocytes were cultured for 4 days on micro-contact printed substrates prior to experimentation.

### Fabrication of Micro-Contact Printed Substrates

Silicone stamps designed for micro-contact printing were prepared. Photolithographic masks were designed in AutoCAD (Autodesk Inc., San Rafael, Calif.), and consisted of 20 μm wide lines separated by 4 μm gaps to impose a laminar organization on the myocytes. Polydimethylsiloxane (PDMS, Sylgard 184, Dow Corning, Midland, Mich.) was used to fabricate stamps with the specified pattern. Stamps were incubated with 50 μg/mL FN (BD Biosciences, Bedford, Mass.) for one hour. Glass coverslips were spin-coated with PDMS and treated in a UV-ozone cleaner (Jelight Company, Inc., Irvine, Calif.) immediately prior to stamping with FN. After transfer of the FN pattern to the surface of the PDMS-coated coverslips, they were incubated in 1% (w/v) Pluronic F127 (BASF, Ludwigshafen, Germany) to block cell adhesion to un-stamped regions.

### “Heart-on-a-Chip” Substrate Fabrication

Engineered cardiac tissue contractile performance was measured using a custom muscular thin film based platform. Briefly, the “heart-on-a-chip” substrates consisted of glass coverslips selectively coated with a thermo-sensitive sacrificial polymer, Poly(N-isopropylacrylamide) (PIPAAm, Polysciences, Inc., Warrington, Pa.), and with a second layer of PDMS. The thickness of the PDMS layer was found to be in the range of 10-18 μm for all “heart chips” used in this study (Dektak 6M, Veeco Instruments Inc., Plainview, N.Y.).

### “Heart-on-a-Chip” Contractility Experiments

During contractility experiments, samples were submerged in Tyrode's solution (mM, 5.0 HEPES, 5.0 glucose, 1.8 CaCl₂, 1.0 MgCl₂, 5.4 KCl, 135.0 NaCl, and 0.33 NaH₂PO₄. All reagents were purchased from Sigma Aldrich, St. Louis, Mo.). Rectangular films were cut out with a razor blade, and the bath temperature was decreased below the PiPAAm transition temperature, making possible for the MTF to bend away from the glass. Video recording of the deformation of each film were processed to obtain the time-course (Alford et al., 2010, Biomaterials 31:3613-3621) of the tissue-generated stresses. The peak systolic and diastolic stresses were calculated as the average of the maxima and minima of the stress profile during 10 cycles at a pacing of 3 Hz, and twitch stress was defined as the difference between peak systolic and diastolic stresses.

### Immunohistochemical Labeling

Samples were fixed in 4% (v/v) paraformaldehyde with 0.05% (v/v) Triton X-100 in PBS at room temperature for 10 minutes. Cells were incubated in a solution containing 1:200 dilutions of monoclonal anti-sarcomeric α-actinin antibody (A7811, clone EA-53, Sigma Aldrich, St. Louis, Mo.), polyclonal anti-fibronectin antibody (F3648, Sigma-Aldrich, St. Louis, Mo.), 4′,6′-diamidino-2-phenylindole hydrochloride (DAPI, Invitrogen, Carlsbad, Calif.), and Alexa Fluor 633-conjugated phalloidin (Invitrogen, Carlsbad, Calif.) for one hour at room temperature. Samples were then incubated in 1:200 dilutions of Alexa Fluor 488-conjugated goat anti-mouse IgG and Alexa Fluor 546-conjugated goat anti-rabbit IgG secondary antibodies (Invitrogen, Carlsbad, Calif.) for 1 hour at room temperature. Labeled samples were imaged with a Zeiss LSM confocal microscope (Carl Zeiss Microscopy, Jena, Germany).

### Evaluation of Sarcomere Structure

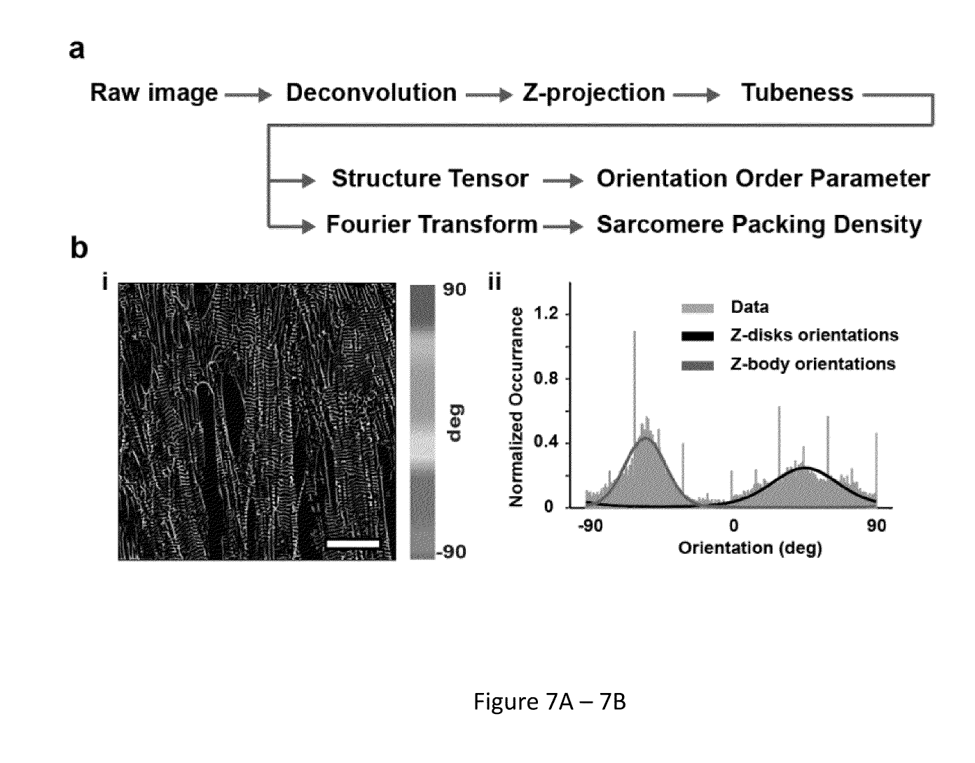

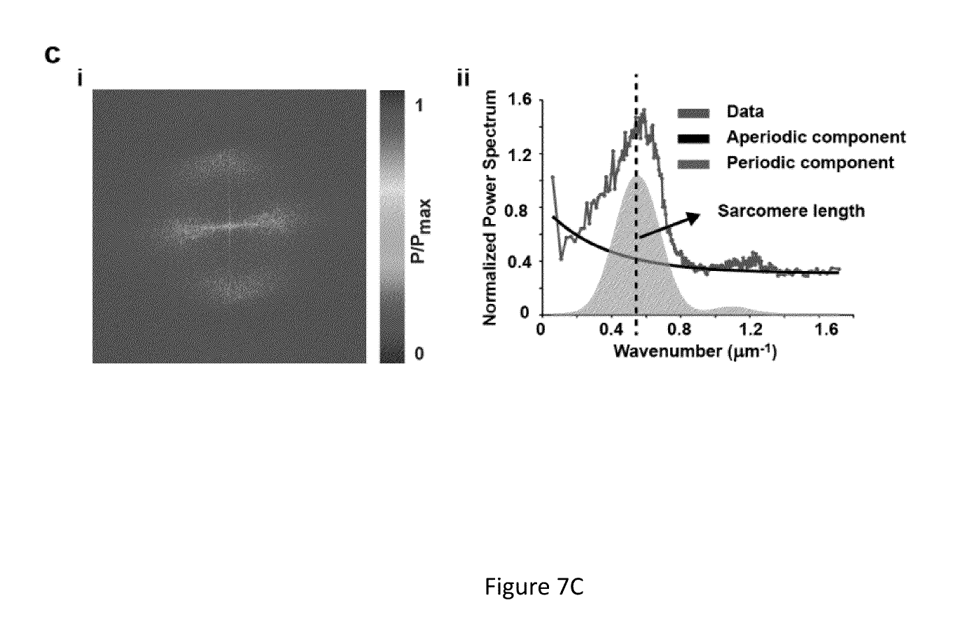

Analysis of sarcomeric structural characteristics was conducted, after de-convolving acquired confocal Z-stacks of sarcomeric α-actinin fluorescence micrographs with Mediacy Autoquant (MediaCybernetics, Rockville, Md.), on custom-designed ImageJ (NIH) and MATLAB (Mathworks, Natick, Mass.) software. Fluorescence micrographs were first pre-processed to highlight the filamentous structure of the cytoskeleton using a “tubeness” operator. This operator replaced each pixel in the image with the largest non-positive eigenvalue of the image Hessian matrix. The orientations of sarcomeric α-actinin positive pixels were determined using an adapted structure-tensor method and the orientational order parameter (OOP), a measure of the global alignment of the sarcomeres, was calculated from the observed orientations. The orientations observed in the micrographs were color-coded using the HSV digital image representation (FIG. 7B(i)) where the Hue channel was used for orientation, the Saturation channel for pixel coherency (i.e. a measure of local contrast), and the Value channel for the pre-processed image. The normalized occurrence of the orientations that demonstrated a coherency higher than a given threshold (sub-threshold pixels were not color-coded) could then be displayed in a histogram (FIG. 7B(ii)). Two components could be easily distinguished: blue-green coloration in (FIG. 7B(i)) corresponded to pixels localized to Z-disks (black curve in FIG. 7B(ii)), while red-yellow pixels were associated with long stretches of Z-bodies (red curve in FIG. 7B(ii)). The sarcomere length and the overall regularity of the z-lines was determined by processing the fluorescence images with a 2D Fast Fourier Transform algorithm (the power spectrum of the image in FIG. 7B(i) is reported in FIG. 7C(i) with a gamma correction of 0.1 to improve visualization). To further analyze the Fourier representation without introducing user-bias, the power spectrum was then radially integrated and normalized by the total area under the 1D curve. The previous step yielded a 1D profile (blue curve in FIG. 7C(ii)) that could be fitted with aperiodic (3, black line in FIG. 7C(ii)) and periodic (red line in FIG. 7C(ii)) components. The parameters {a, b, c} in (1) characterize the decaying exponential chosen to model the effect of noise and non-regularly distributed structures in the image, while the parameters {ω₀, αₖ, δₖ} in (2) represent respectively, the wavenumber that corresponds to the sarcomere length, the amplitude and the width of the Gaussian peaks chosen to model the periodic peaks. The sarcomere packing density was defined as the area under the periodic component (shaded red in FIG. 7C(ii)).

$$\begin{matrix}{\tilde{Γ}}_{ap} ( ω , {γ}_{ap} ) = a + b {e}^{- c ω} ; \\ {γ}_{ap} = \{ a , b , c \} & ( 1 ) \\ {\tilde{Γ}}_{p} ( ω , {γ}_{p} ) = {\sum }_{k = 1}^{3} {a}_{k} {e}^{- {( \frac{ω - k {ω}_{0}}{{δ}_{k}} )}^{2}} ; \\ {γ}_{ap} = \{ {ω}_{0} , {a}_{k} , {δ}_{k} \} & ( 2 )\end{matrix}$$

### Planar Patch Clamp Electrophysiological Recordings

Planar patch clamp experiments were conducted as previously described. Briefly, cells were cultured on fibronectin (BD Biosciences, Bedford, Mass.) coated T25 flasks for 5 days, then isolated using 0.25% trypsin (Invitrogen, Carlsbad, Calif.), re-suspended in Extracellular Buffer Solution (EBS: mM, 140 NaCl, 4 KCl, 1 MgCl₂, 2 CaCl₂, 5 D-Glucose monohydrate, 10 Hepes, pH 7.4) to a final concentration of 1,000 cells/μL, and allowed to equilibrate for 5 minutes in EBS. The electronics were calibrated in the presence of EBS and Intracellular Buffer Solution (IBS: mM, 50 KCl, 10 NaCl, 60 K-Fluoride, 20 EGTA, 10 Hepes, pH 7.2) prior to flowing cells into the chamber. 5 μL of cell suspension was then introduced into the chip and the negative pressure automatically adjusted to produce a final seal resistance greater than 1 GOhm. During current clamp experiments, cells were subjected to 10 trains of 10 current pulses at 3 Hz; the current amplitude was set to 1.5 times the threshold for Action Potential (AP) generation. When the signal reached steady state, 10 APs were averaged yielding a representative trace for the calculation of action potential duration indicators. During voltage clamp experiments cells were kept in buffers containing TTX (10 μM), Nifedipine (10 μM), 4-AP (1 mM) and TEA (20 mM) purchased from Sigma Aldrich (St. Louis, Mo.). The membrane potential subjected to 2 voltage clamp protocols, first the membrane potential was held to a value of −90 V for 250 ms and then stepped from −70 to +40 mV in 10 mV steps for 250 ms, thus eliciting the total Ca²⁺ current (TOT). Second, from the same holding potential, cells were stepped from −40 to +40 mV, a range in which mostly the L-type Ca²⁺ current (LCC) is active. The T-type component (TCC) was then calculated as the difference between TOT and LCC.

### Optical Mapping of Electrophysiological Properties

Samples were incubated in 4 μM RH237 (Invitrogen, Carlsbad, Calif.) for 5 minutes and washed 3 times with Tyrode's solution, prior to recording. Temperature of the bath solution was maintained at approximately 35° C. using a digital temperature controller (TC-344B, Warner Instruments, Hamden, Conn.) for the duration of the experiment. 10 μM Blebbistatin (EMD Millipore, Billerica, Mass.) was added to minimize motion artifacts during recording of electrical activity. Samples were paced at 3 Hz with a 10 ms biphasic pulse at 10-15 V delivered using an SD-9 stimulator (Grass Technologies, Warwick, R.I.) and a bi-polar, platinum point electrode placed approximately 300-500 μm above the sample and 1-2 mm from the top right corner of the field of view (FOV). Imaging was performed using a Zeiss Axiovert 200 epifluorescence microscope (Carl Zeiss Microscopy, Jena, Germany) equipped with an X-cite Exacte mercury arc lamp (Lumen Dynamics, Mississauga, Ontario). Illumination light was passed through a 40×/1.3 NA objective (EC Plan-NEOFLUAR, Zeiss, Jena, Germany) and a band-pass excitation filter (530-585 nm). Emission light was filtered at 615 nm with a long-pass filter, and focused onto the 100×100 pixel chip of a high speed MiCAM Ultima CMOS camera (Scimedia, Costa Mesa, Calif.). Images were acquired at 1000 frames per second from 250×250 μm fields of view. Post-processing of the raw data included reduction of drift induced by photobleaching by subtracting a linear fit of the baseline, applying a 3×3 pixel spatial filter to improve signal to noise ratio, and exclusion of saturated pixels. Activation time was calculated as the average maximum upstroke slope of multiple pulses over a 2-4 second recording window. Longitudinal and transverse conduction velocities (LCV and TCV) were calculated through a linear fit of the activation times along the horizontal and vertical axes of each FOV respectively. Optical action potential traces were calculated as the average of multiple pulses, while adjusting the offset of each pixel caused by different activation times.

### Ratiometric Measurement of Cardiomyocyte Calcium Transients

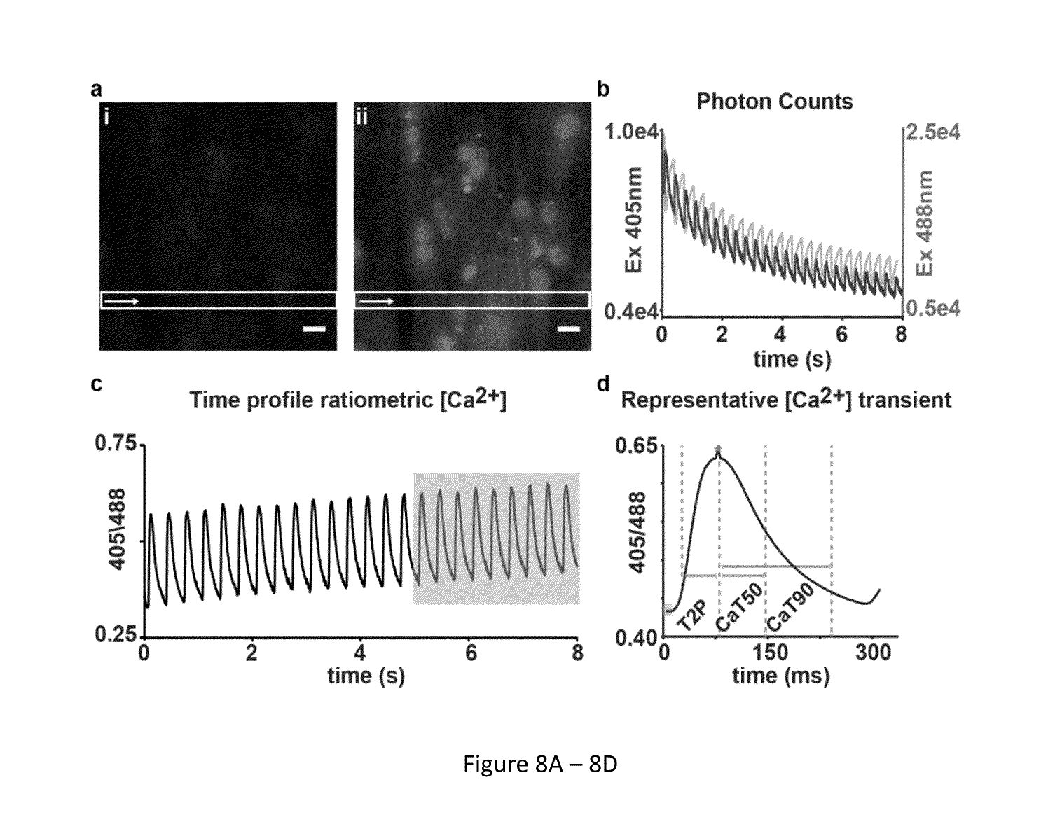

20 μL working aliquots of acetoxymethyl (AM) Fura Red (Invitrogen, F-3021) were obtained reconstituting 50 μg of the desiccated dye in 100 μL of Pluronic F-127 (20% solution in DMSO; Invitrogen, P-3000MP). Working aliquots were stored in the freezer and used within the week. Dye loading of myocytes was performed by exposing the cells for 20 minutes to a solution composed from a single working aliquot diluted in 2 mL of media. After dye loading, cells were kept in Tyrode's solution for 5 minutes, washed 3 times, and mounted on a coverslip holder for confocal imaging. Tissues were imaged using a Zeiss LSM LIVE (Carl Zeiss Microscopy, Jena, Germany) confocal microscope and a 40× objective equipped with an environmental chamber to ensure a constant physiological temperature in the bath of 37° C. Tissues were field stimulated at 3 Hz using the same equipment adopted in MTF experiments. Dual excitation ratiometric recordings were performed by rapidly switching (through an acousto-optical tunable filter) excitation laser lights at 405 nm and 488 nm and by collecting the corresponding emissions through a high-pass filter with cutoff at 546 nm. The 405 nm excitation offers an estimated 16% higher absorbance than what was recently reported for a 457 nm excitation light, while reducing the overlap between the Ca²⁺-bound and Ca²⁺-free excitation spectra. To maintain a high enough acquisition speed (250 fps), the recordings were constrained to 20 lines, oriented perpendicular to the main axis of the cells and ensuring minimal intersection with nuclei (white box FIG. 8A). After background subtraction (performed in FIJI⁴²), two signals were obtained (FIG. 8B): one (blue line) that increases with the Ca²⁺ elevation corresponding to excitation at 405 nm, and one (green line) that shows an opposite trend and corresponded to the 488 nm excitation wavelength. The ratiometric representation of the calcium transient was taken as the ratio of the 405 nm and 488 nm signals (black trace in FIG. 8C). Four consecutive transients at steady state were further averaged to create a representative single transient (FIG. 8D) that was used to extract the following quantities: diastolic level (grey box), peak level (*), time to peak (T2P) and the duration of the Ca²⁺ transient at 50% (CaT50) and 90% (CaT90) decay using Matlab (Mathworks, Natick, Mass.).

### RT-qPCR Gene Expression Measurements

Total RNA was collected in triplicate from both isotropic and micropatterned anisotropic samples using a Strategene Absolutely RNA Miniprep kit (Agilent Technologies, Santa Clara, Calif.) according to the manufacturer's instructions. Genomic DNA contamination was eliminated by incubating the RNA lysates in DNase I digestion buffer at 37° C. for 15 minutes during the RNA purification procedure. The quantity and purity of RNA lysates was assessed using a Nanodrop spectrophotometer (Thermo Scientific, Wilmington, Del.). Purified total RNA lysates with OD 260/280 ratios greater than 1.7 were used for RT-qPCR measurements. Complementary DNA strands were synthesized for genes of interest using an RT2 first strand synthesis kit (Qiagen Inc, Valencia, Calif.) and custom pre-amplification primer sets (Qiagen Inc, Valencia, Calif.). 500 ng of total RNA were used from each lysate for each first strand synthesis reaction. Expression levels for specific genes of interest (Table 3 and Table 4) were measured using custom RT2 Profiler RT-PCR arrays (Qiagen Inc, Valencia, Calif.) and a Bio-Rad CFX96 RT-PCR detection system (Hercules, Calif.). Statistical analysis of RT-qPCR threshold cycle data was carried out with the web-based RT2 Profiler PCR Array Data Analysis Suite version 3.5 (Qiagen Inc, Valencia, Calif.) according to published guidelines.

### Statistical Analysis

All data are summarized as mean±standard error of the mean. Data were first tested for normality (Shapiro-Wilk) and equal variance (Levene Median test). Based on the results from these tests, either 1-way ANOVA or ANOVA on Ranks were adopted to establish statistical difference between the groups. Pairwise comparisons were then assessed using either Dunn's or Tukey or Holm-Sidak methods as post-hoc tests. In the figures the significance of statistical tests (p-value) is indicated as follows: *=p<0.05, **=p<0.001 for 1-way ANOVA and fort=p<0.05, if =p<0.001 ANOVA on ranks.

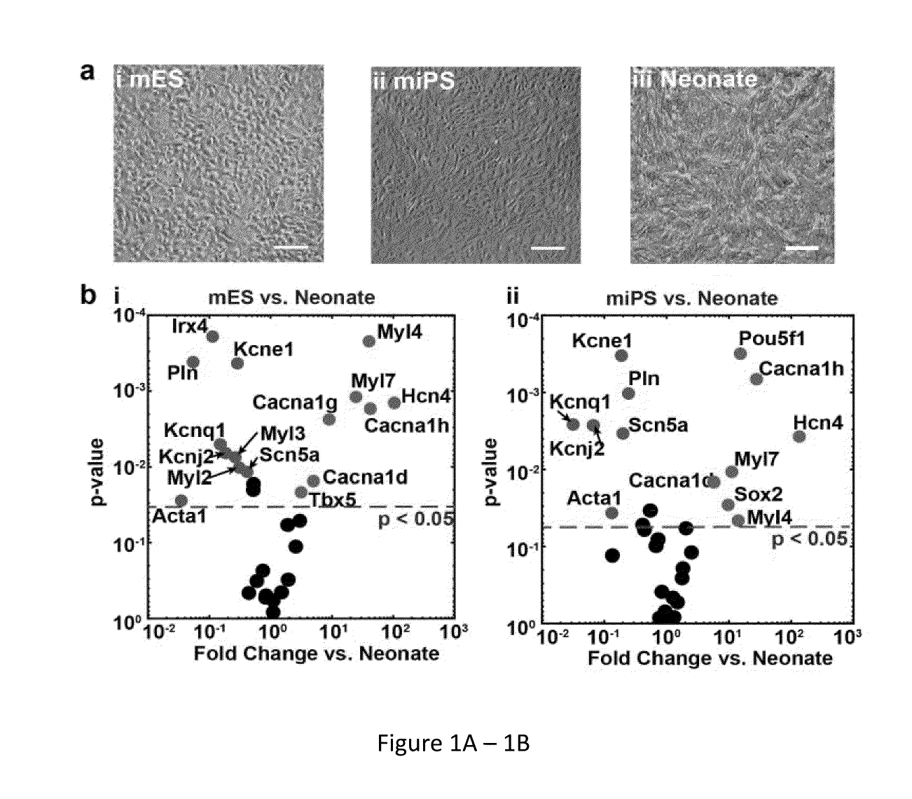

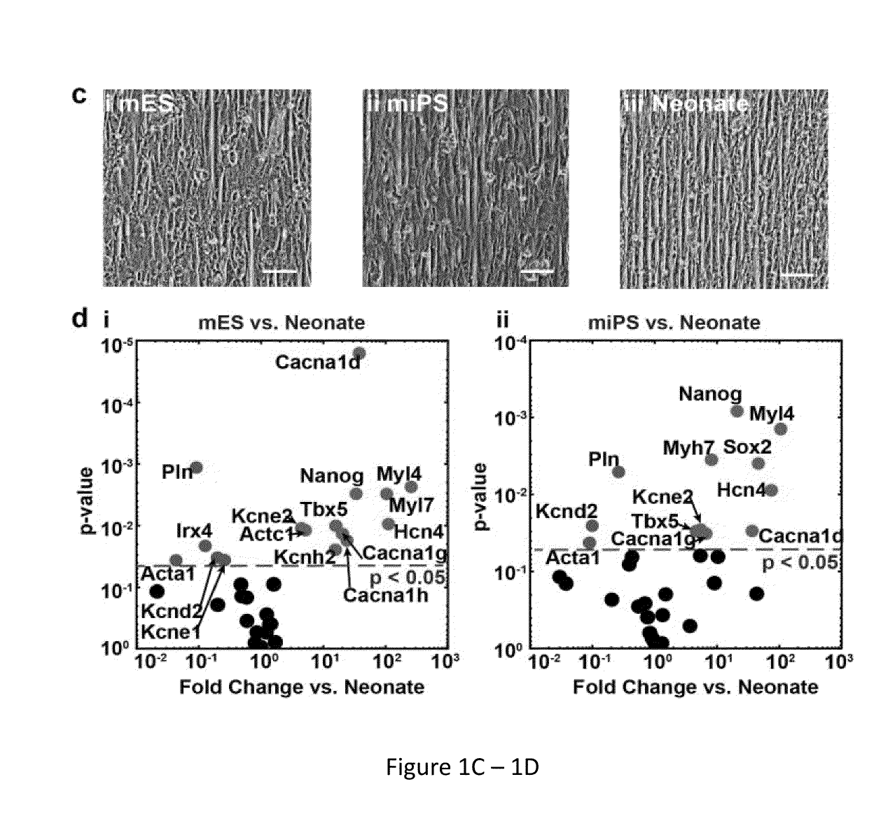

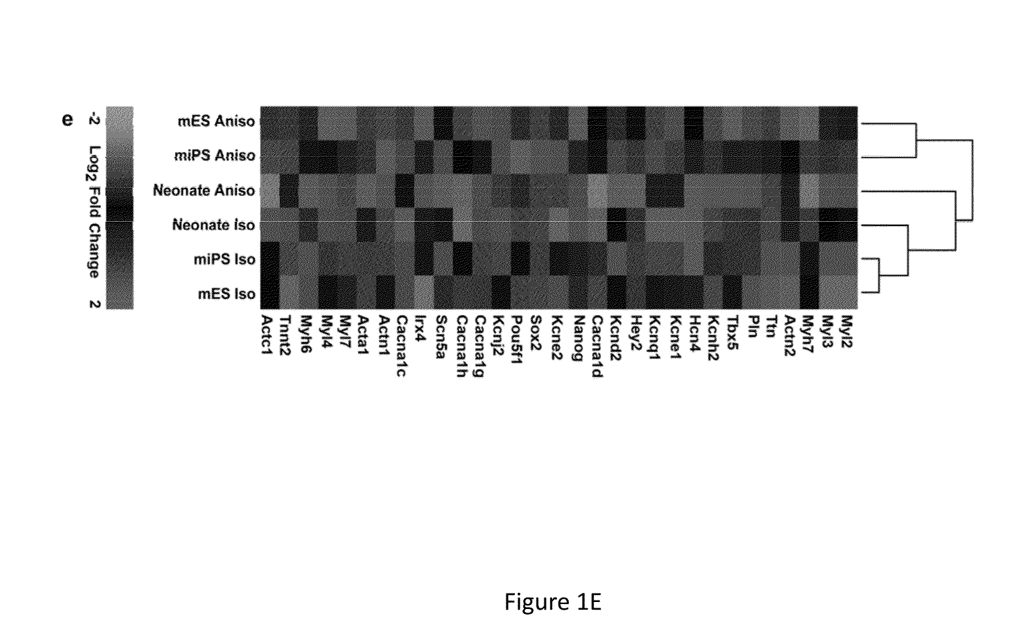

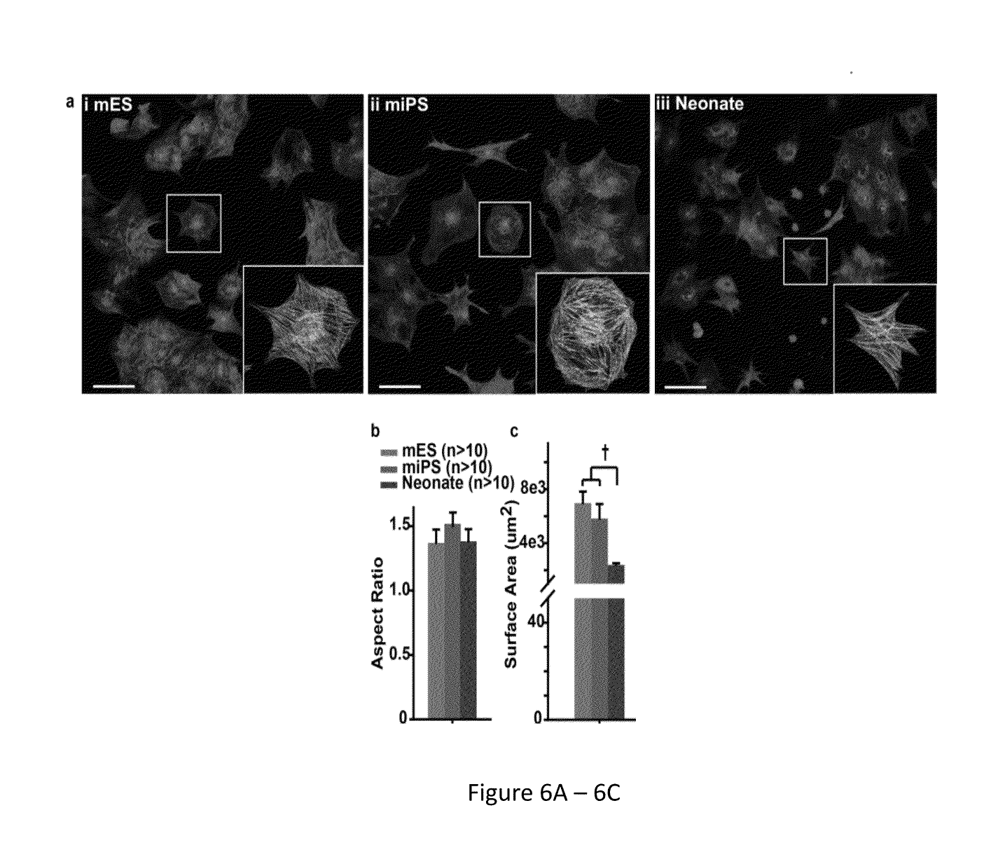

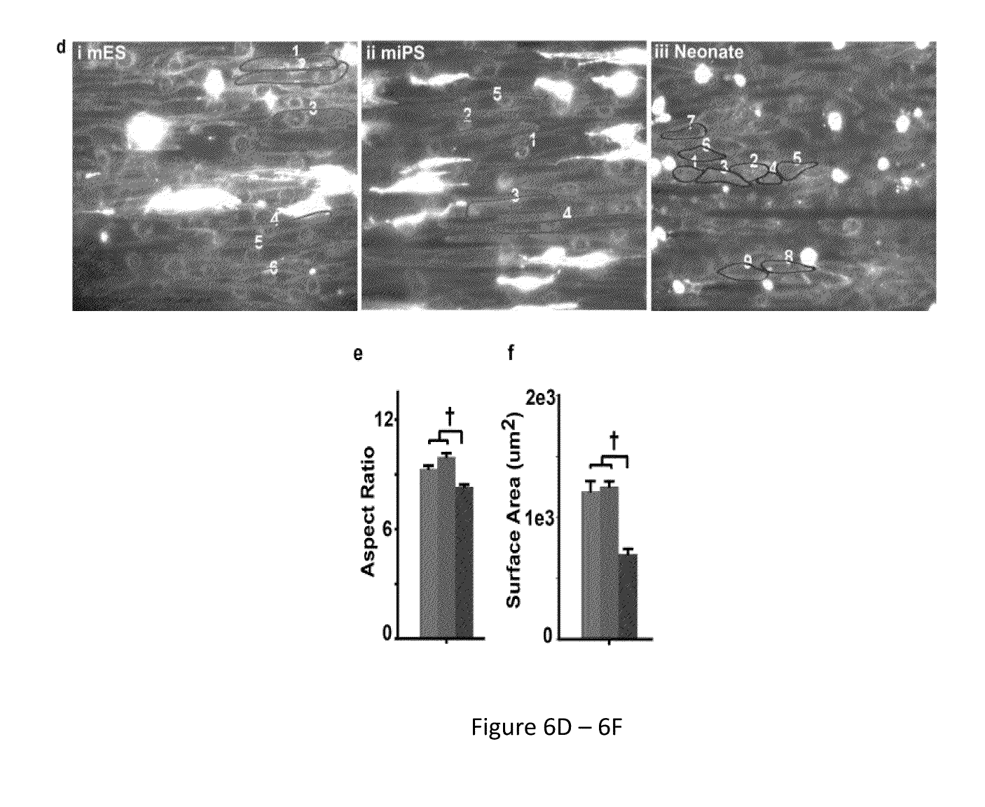

The influence of tissue architecture on the contractile performance of engineered myocardium in vitro was previously reported (Feinberg et al., 2012, Biomaterials 33:5732-5741). From this, characterization of the mES and miPS myocytes is made by evaluating their response to geometric cues encoded in the ECM, and measuring the expression of genes that are commonly used to delineate the cardiac myocyte lineage (Maltsev et al., 1994, Circ Res 75:233-244; Chin et al., 2009, Cell Stem Cell 5:111-123; Sartiani et al., 2007, Stem Cells 25:1136-1144). Culturing mES (FIG. 1A(i)) and miPS (FIG. 1A(ii)) myocytes on a substrate coated uniformly with fibronectin (FN) gave rise to monolayers with an isotropic cellular arrangement similar to the arrangement observed when neonate ventricular myocytes (FIG. 1A(iii)) were cultured in a similar manner. Moreover, mES (FIG. 6A(i)), and miPS (FIG. 6A(ii)) and neonate (FIG. 6A(iii)) myocytes all assumed a pleomorphic morphology when cultured sparcely on isotropic FN (FIG. 6B), even though the neonate cardiac myocytes displayed a smaller surface area than the mES and miPS myocytes (FIG. 6C). Comparison of the expression profiles for isotropic mES (FIG. 1B(i)) and miPS (FIG. 1B(ii)) derived tissues versus the neonate tissues revealed a number of significant differences associated with ion channel subunits and components of the sarcomere. In particular, the mES tissues exhibited significantly higher expression of the L-type Ca²⁺ channel subunit Cacnald (4.9 fold, p<0.05), as well as the T-type subunits Cacnalg (9.0 fold, p<0.05) and Cacnalh (42.2 fold, p<0.05) versus neonate tissues. Isotropic mES tissues also showed significantly lower expression of Irx4 (−9.1 fold, p<0.001), Myl2 (−3.2 fold, p<0.05), and Myl3 (−3.8 fold, p<0.01) commonly associated with the ventricular myocyte phenotype (Ng et al., 2010, Am J Physiol Cell Physiol 299:C1234-1249), and significantly higher expression of the atrial marker genes My14 (40.2 fold, p<0.001), and My17 (24.5 fold, p<0.01) than the neonate isotropic tissues. In contrast, the miPS isotropic tissues showed significant differences in expression for Cacnald (5.7 fold, p<0.05), Cacnalh (27.9 fold, p<0.001), My14 (14.1 fold, p<0.05) and My17 (11.1, p<0.05) versus the neonate isotropic tissues. These observations suggest that the miPS-derived myocytes exhibited an expression profile that more closely resembled the profile of the neonate ventricular myocytes than the mES-derived myocytes.

Based on previous studies, it was recognized that the gene expression profile of cardiac myocytes changed as a function of the tissue architecture within which they are embedded. Laminar, anisotropic myocardium was engineered from mES (FIG. 1C(i)), miPS (FIG. 1C(ii)), and neonate cardiac myocytes by culturing them on micro-contact printed FN, where the cells spontaneously formed cell-cell junctions and aligned with the geometric cues within the matrix to form a contiguous tissue of high aspect ratio cells (FIG. 6D and FIG. 6E). After several days in culture, the expression profiles of these engineered tissues were measured and compared. Comparison of the expression profiles for anisotropic neonate and mES tissues (FIG. 1D(i)) revealed a number of differences associated with Ca²⁺ channel subunits, such as the L-type Ca²⁺ channel subunit Cacnald (37.5 fold, p<0.0001), as well as the T-type subunits Cacnalg (20.2 fold, p<0.05), and Cacnalh (23.8 fold, p<0.05). Additionally, the mES anisotropic tissues showed significantly lower expression of the ventricular marker Irx4 (−7.7 fold, p<0.05), and significantly higher expression of the atrial markers My14 (254.8 fold, p<0.01), and My17 (104.0 fold, p<0.01) versus the neonate tissues. In contrast, the miPS anisotropic tissues exhibited significant differences from the neonate tissues (FIG. 1D(ii)) for the Ca²⁺ channel subunits Cacnald (36.9 fold, p<0.05) and Cacnalg (6.6 fold, p<0.05), as well as the atrial myosin light chain kinase gene My14 (105.5 fold, p<0.01). Hierarchical clustering of neonate, mES, and miPS gene expression measurements revealed a distinct separation of the expression profiles for isotropic and anisotropic tissues, regardless of myocyte type (FIG. 1E). Moreover, the expression profiles for mES and miPS myocytes in both the isotropic and anisotropic cellular configurations clustered closer to each other than to the neonate tissues, suggesting that the mES and miPS myocytes exhibited global transcriptional profiles that were unique from the neonate expression pattern, despite differences in the relative expression profiles between the mES and miPS tissues.

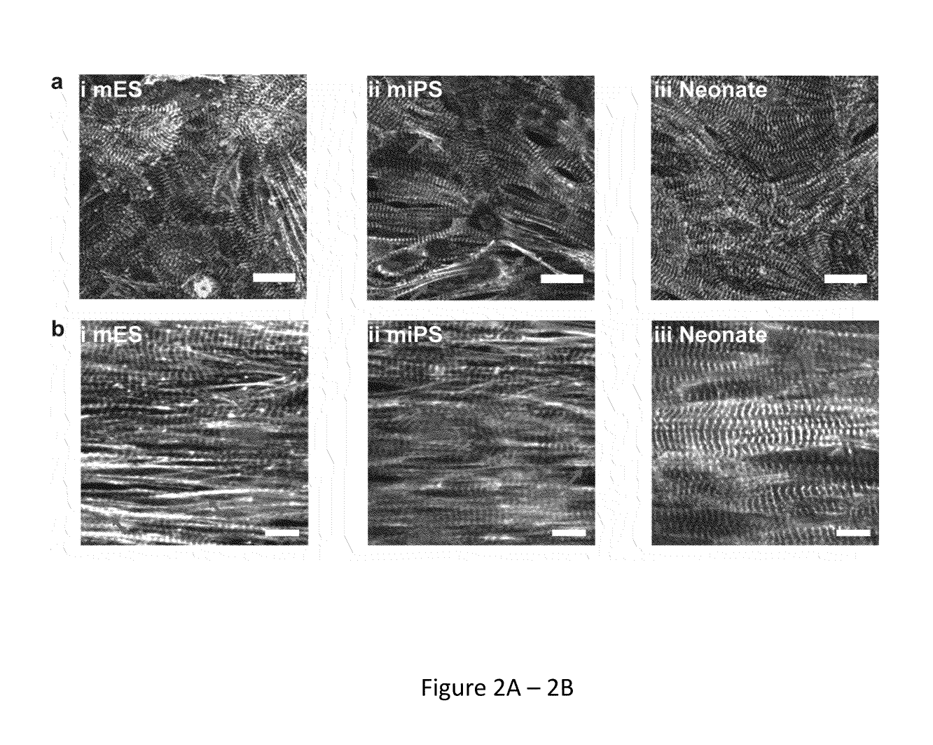

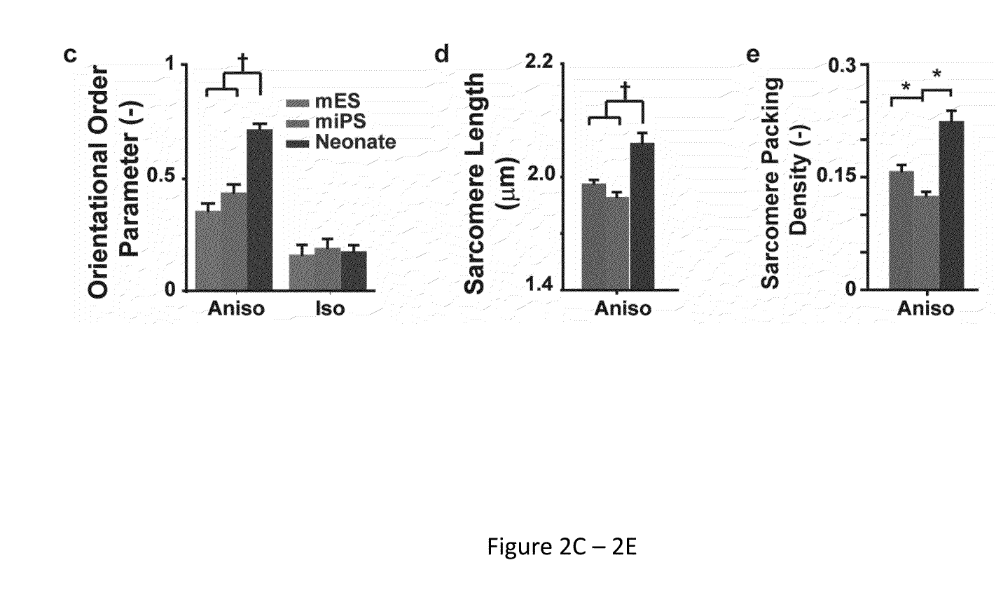

One of the defining features of the native myocardium is the laminar arrangement of cardiac myocytes that serves to organize and orient the contractile sarcomeres to facilitate efficient pump function (Bruneau, 2002, Circ Res 90:509-519). The ability of mES and miPS engineered tissues to self-assemble myofibrils with alignment comparable to neonate ventricular myocytes were evaluated using image analysis software of the present invention. Immunofluorescence micrographs of sarcomeric α-actinin allowed for visualization of the orientations of the z-lines outlining the lateral edges of sarcomeres and to quantitatively assess sarcomere organization in the engineered tissues. Visualization of global z-line registration in isotropic monolayers of mES (FIG. 2A(i)), miPS (FIG. 2A(ii)) and neonate (FIG. 2A(iii)) myocytes revealed random orientation patterns. In contrast, the anisotropic mES (FIG. 2B(i)), miPS (FIG. 2B(ii)), and neonate (FIG. 2B(iii)) tissues demonstrated a greater degree of uniaxial z-line registration. To quantify the differences in global sarcomere organization between the mES, and miPS tissues, versus the neonate tissues (FIG. 2C), a metric known as the orientational order parameter (OOP) was utilized, which is commonly used to characterize the alignment of liquid crystals, and ranges from zero (random organization) to one (perfect alignment). The anisotropic neonate tissues exhibited a significantly higher OOP value than both the mES and miPS tissues, suggesting that both types of stem cell-derived myocytes were unable to generate myofibrils with the same degree of global sarcomere alignment as the neonate myocytes. Isotropic tissues had low OOP values, due to the random organization of the cardiac myocytes. Measurement of registered z-line spacing also revealed that the anisotropic mES and miPS tissues displayed significantly shorter sarcomere lengths than the neonate tissues (FIG. 2D). Moreover, quantification of “sarcomere packing density,” i.e. the proportion of α-actinin localized to z-lines indicative of the presence of fully-formed sarcomeres, showed that the anisotropic neonate tissues exhibited significantly higher sarcomere packing density than the mES and miPS tissues. Taken together, these analyses revealed that the mES- and miPS-derived myocytes responded to ECM cues in a similar manner to the neonate myocytes, but exhibited sarcomere organization reminiscent of immature pre-myofibrils observed in embryonic cardiac myocytes (Dabiri et al., 1997, Proc Natl Acad Sci USA 94:9493-9498; LoRusso et al., 1997, Cell Motil Cytoskeleton 37:183-198).

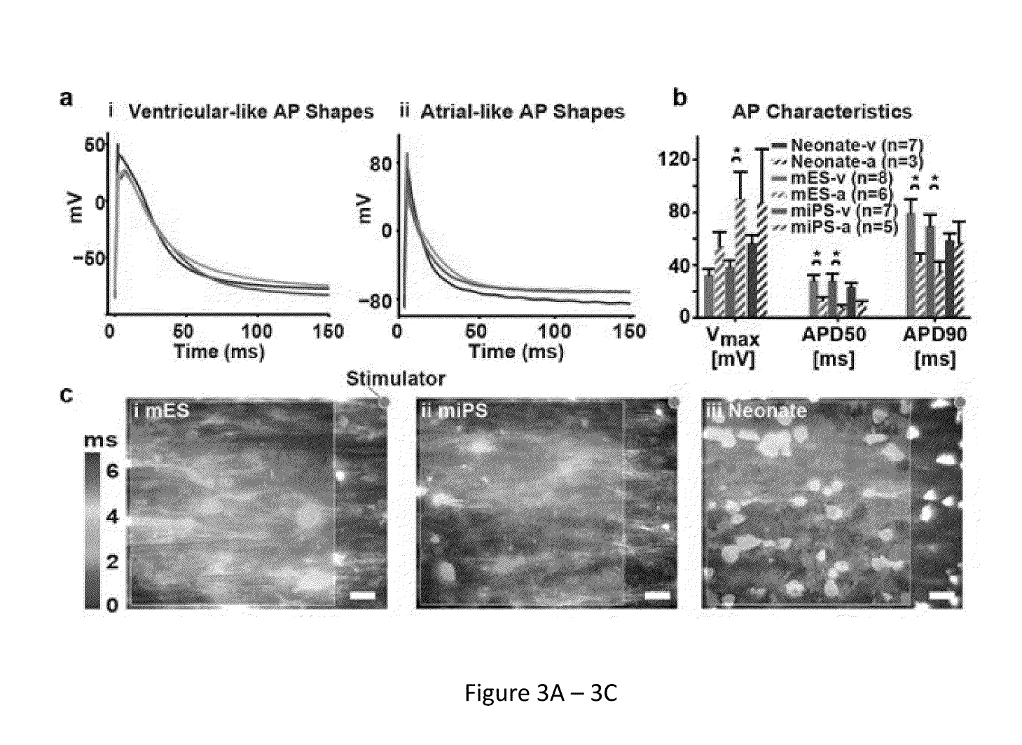

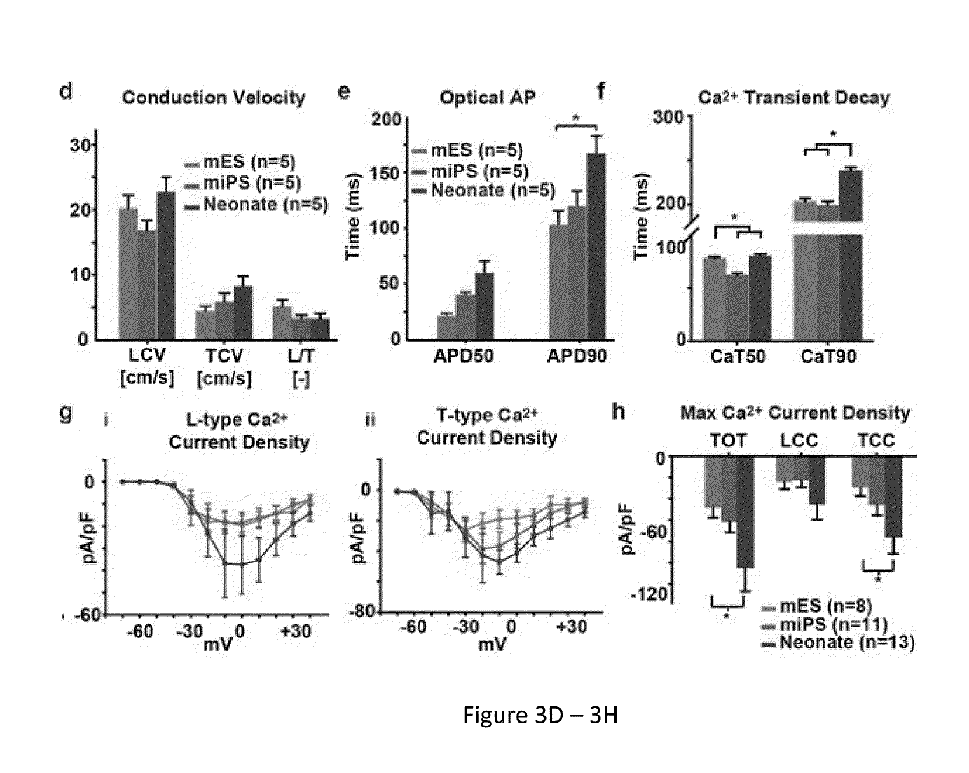

The electrical activity of cardiac myocytes regulates the initiation of myofibril contraction and is commonly measured as an indicator of myocyte identity and functionality (Kleber and Rudy, 2004, Physiol Rev 84:431-488; Maltsev et al., 1994, Circ Res 75:233-244; Weinberg et al., 2010, Methods Mol Biol. 660:215-237). Planar patch clamp recordings were used to compare and contrast the action potential characteristics of isolated mES, miPS and neonate myocytes. Two different demographics of cell types were identified, demonstrated by action potential morphology (AP). Most neonate myocytes mostly demonstrated ventricular-like APs (FIG. 3A(i)) whereas mES- and miPS-derived myocytes exhibited APs that were evenly distributed between the ventricular-like (FIG. 3A(i)) and satrial-like (FIG. 3A(ii)) morphologies. Both the mES- and miPS-derived myocytes primarily exhibited APs as shown in

FIG. 3A(ii), whereas the neonate ventricular myocytes demonstrated APs illustrated in FIG. 3A(i). Analysis of AP characteristics, such as maximum voltage (Vₘₐₓ), action potential duration at 50% repolarization (APD50), and action potential duration at 90% repolarization (APD90), revealed that the mES and miPS myocytes exhibited roughly equal incidences of atrial-like and ventricular-like APs, whereas the neonate cardiac myocytes displayed ventricular-like AP characteristics (FIG. 3B). In addition to AP characterization, the electrical conduction properties of the anisotropic mES (FIG. 3C(i)), miPS (FIG. 3C(ii)), and neonate (FIG. 3C(iii)) tissues were measured using optical mapping and the voltage-sensitive fluorescent dye RH-237 (Weinberg et al., 2010, Methods Mol Biol. 660:215-237; Bursac et al., 2002, Circ Res 91:e45-54; Thomas et al., 2000, Circ Res 87:467-473) to evaluate the ability of the stem cell-derived myocytes to form the electromechanical syncytium that typifies the myocardium (Kleber and Rudy, 2004, Physiol Rev 84:431-488). No significant differences in the longitudinal (LCV) or transverse (TCV) conduction velocities were observed between the mES, miPS and neonate tissues (FIG. 3D). AP duration measurements revealed no significant differences at 50% repolarization (APD50), but a significant (p<0.05) difference was observed at 90% repolarization (APD90) between the neonate and mES anisotropic tissues (FIG. 3E). Ca²⁺ plays a crucial role in coupling myocyte excitation and contractile activity (Bers, 2002, Nature 415:198-205), therefore, the Ca²⁺ transient activity in engineered anisotropic tissues, as well as the Ca²⁺ current profiles of isolated mES, miPS and neonate myocytes were measured. Ca²⁺ transients measured in anisotropic tissues revealed a significantly (p<0.05) shorter 50% decay time (CaT50) in the miPS, but not the mES tissues, as compared to the neonate, and significantly (p<0.05) shorter 90% decay time (CaT90) in both the mES and miPS tissues versus the neonate tissues (FIG. 3F). Planar patch clamp recordings of L- (FIG. 3G(i)) and T- (FIG. 3G(ii)) type Ca²⁺ current profiles revealed significantly (p<0.05) higher total (TOT) and T-type (TCC) maximum Ca²⁺ current densities in the neonate myocytes versus the mES-derived, but not the miPS-derived myocytes (FIG. 3H). Taken together, these data suggest that the mES and miPS myocytes possessed electrophysiological properties similar to neonate cardiac myocytes, aside from differences in funny current and voltage-gated Ca²⁺ channel subunit expression illustrated in FIG. 1.

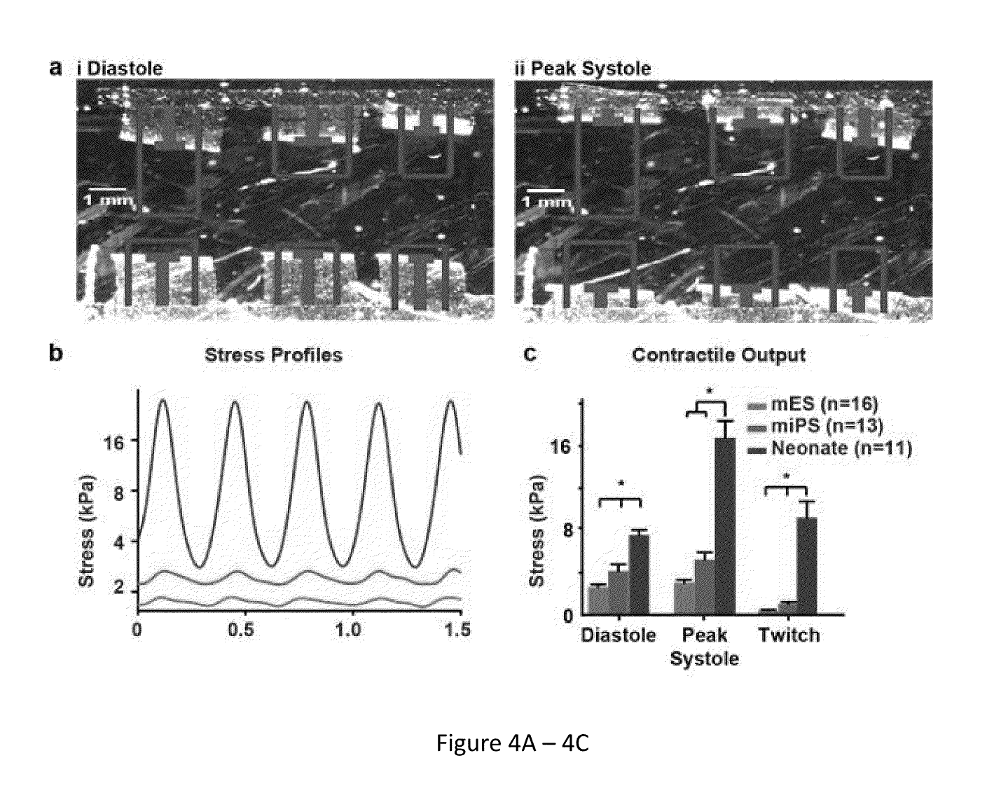

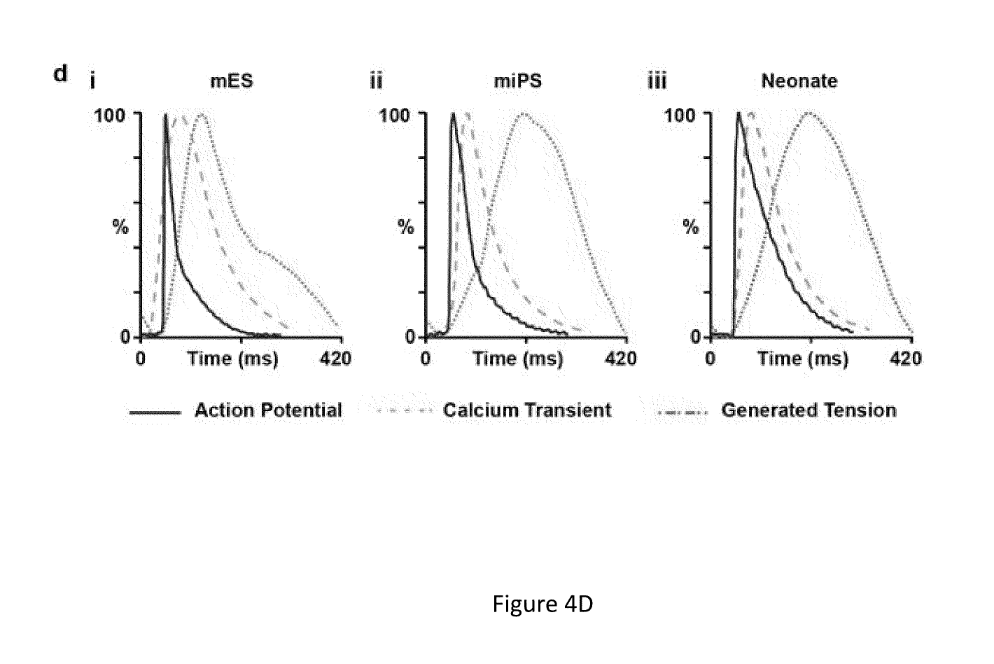

With the muscular thin film (MTF) contractility assay, it is now possible to assess the diastolic (FIG. 4A(i)) and systolic (FIG. 4A(ii)) function of engineered myocardium directly (Alford et al., 2010, Biomaterials 31:3613-3621; Grosberg et al., 2011, Lab Chip 11:4165-4173; Feinberg et al., 2007, Science 317:1366-1370). Using the “heart-on-a-chip” MTF assay, the stress generation profiles of the anisotropic mES, miPS and neonate tissues were measured (FIG. 4B), and their contractile performance compared. The anisotropic neonate tissues generated significantly (p<0.05) higher diastolic, peak systolic, and twitch stress than both the mES and miPS tissues (FIG. 4C), with observed values for the neonate tissues within the range measured for isolated murine papillary muscle strips (Stuyvers et al., 2002, J Physiol 544:817-830; Kentish et al., 2001, Circ Res 88:1059-1065; Gao et al., 1998, J Physiol 507(Pt 1):175-184). The results of the contractility measurements clearly show a functional deficit in the mES- and miPS-derived myocytes that was not apparent in the electrophysiological measurements. The combined output of the electrophysiological, calcium transient and contractile force experimental measurements were used to create graphical representations of the excitation-contraction coupling profiles of the mES (FIG. 4D(i)), miPS (FIG. 4D(ii)), and neonate (FIG. 4D(iii)) engineered tissues that clearly illustrate the similarities and differences in the excitation-contraction coupling amongst the cell types. These data illustrate that the miPS-derived myocytes are qualitatively more similar to the neonate myocytes than the mES-derived myocytes.

To determine how closely the mES- and miPS-derived myocytes matched the phenotype of the neonate ventricular myocytes, a novel numerical method was developed to integrate the set of gene expression, morphology, electrophysiology, and contractility experimental measurements collected on each cell population, and calculate the difference between the unknown and target cell populations. For each experimental measurement, the values were normalized to the interval [0,1] and calculated the strictly standardized mean difference (β) (Zhang, 2007, Genomics 89:552-561) between each unknown population (i.e. mES, miPS) and the neonate target population as follows:

$$β = \frac{{μ}_{1} - {μ}_{2}}{\sqrt{{σ}_{1}^{2} + {σ}_{2}^{2}}} \qquad ( 3 )$$

where μ represents mean and σ represents standard deviation, to evaluate the magnitude of difference, taking into account the variance in the measurements, between the stem cell-derived myocytes and the neonate cardiac myocytes (FIG. 5). This allowed for determination of the effect size for each experimental measurement when comparing the mES and miPS to the neonate tissues, and to identify the parameters that show the greatest degree of similarity and difference from the target neonate ventricular myocyte tissues.

The β values were then used from each experimental measurement for the mES and miPS tissues and the mean squared error (MSE) versus the neonate tissues was calculated as follows:

$$MSE = \frac{1}{n} {\sum }_{i = 1}^{n} {β}_{i}^{2} \qquad ( 4 )$$

where n is the total number of experimental measurement β values included in the calculation, to evaluate the differences observed for each measurement category (i.e. the β values for gene expression, morphology, electrical activity, contractility used to calculate category-specific MSE values), as well as define a single MSE value calculated from all of the experimental measurements from all categories combined, that represents the total difference between the stem cell-derived and neonate cardiac myocytes based on the measurements performed (Table 1). The strictly standardized mean difference (β) values computed for each experimental measurement were used to calculate mean squared error (MSE) values for each of the major measurement categories, as well as all of the measurements combined, in the comparisons of the mES (MSE_{mES}), and miPS (MSE_{miPS}) engineered tissues to the neonate engineered tissues.

TABLE 1
mean squared error values calculated for each group of
measurements in the comparison of the mES- and miPS-derived
myocytes to the neonate ventricular myocytes.

| Measurement Category | MSE_{mES} | MSE_{miPS} |
|---|---|---|
| Gene Expression | 5.69 | 4.25 |
| Morphology | 1.30 | 1.48 |
| Electrophysiology | 1.16 | 0.57 |
| Contractility | 6.32 | 2.95 |
| All Measurements | 4.95 | 3.60 |

A lower MSE value indicates a better match to the neonate target phenotype, with an MSE value of zero indicating a perfect match.

It was found that the miPS tissues exhibited lower MSE values than the mES tissues for every measurement category, except morphology. In addition, the overall MSE values calculated from all of the experimental measurements combined revealed a lower MSE for the miPS engineered tissues than those comprised of mES-derived myocytes. This suggests that the miPS-derived myocytes exhibited a global phenotype that was slightly closer to the neonate cardiac myocytes than the mES-derived myocytes, although both the mES- and miPS-derived myocytes demonstrated substantial differences from the neonate cardiac myocytes for a number of characteristics.

Descriptions for each abbreviation listed in the right-hand column of FIG. 5 can be found in Table 2. Provided in Table 4 are descriptions of the experimental measurements used for the above calculation, the means and standard deviations for these measurements used in the calculation, and each step in the process of arriving at the β values identified in FIG. 5. The descriptions of the measurements and genes listed in Table 4 are provided in Table 2. The steps of the calculation proceed along the columns from left to right, where the last two columns contain the final β values for each experimental parameter. At the bottom of last two columns of Table 4 is the MSE calculation, representing the quality index “score” for each of the two stem cell-derived myocyte cell lines that were tested.

Accordingly, a quality control standard rubric for assessing stem cell-derived cardiac myocytes is shown. Using the experimental measurements described above and isolated neonatal ventricular myocytes as the reference phenotype, a “quality index” was developed that utilizes the magnitude and variance of these measurements to provide a numeric “score” of how closely the stem cell-derived myocytes match the characteristics of the neonatal cardiac myocytes. The combination of gene expression, morphological evaluation, electrophysiological, and contractility measurements employed allow a user of the system and method of the present invention to pin-point specific differences in the structural and functional properties of the mES and miPS engineered tissues versus the neonate tissues that have important implications for their utility in in vitro assays. Further, this “quality index” not only allows researchers to identify the commercial stem cell-derived myocyte product lines that are most suitable for their needs, it serves the stem cell industry as a quality assurance system for ensuring that batches released to customers faithfully recapitulate the desired phenotype.

TABLE 2
List of major experimental measurement categories.

| Measurement Class | Measurement | Measurement Description |
|---|---|---|
| contractility | Diastolic | Diastolic stress |
| contractility | Systolic | Systolic stress |
| contractility | Twitch | Twitch Stress (Systolic - Diastolic) |
| electrophysiology | LCV | Longitudinal conduction velocity |
| electrophysiology | TCV | Transverse conduction velocity |
| electrophysiology | AR | Anisotropy ratio |
| electrophysiology | APD50 | Action potential duration at 50% repolarization |
| electrophysiology | APD90 | Action potential duration at 90% repolarization |
| electrophysiology | TOT | Total calcium current density |
| electrophysiology | LCC | L-type calcium current density |
| electrophysiology | TCC | T-type calcium current density |
| morphology | SPD | Sarcomere packing density |
| morphology | SL | Sarcomere length |
| morphology | OOP | Orientational order parameter |
| gene expression | Hey2 | Hairy/enhancer-of-split related with YRPW motif 2 |
| gene expression | Irx4 | Iroquois related homeobox 4 (Drosophila) |
| gene expression | Gata4 | GATA binding protein 4 |
| gene expression | Myocd | Myocardin |
| gene expression | Nkx2-5 | NK2 transcription factor related, locus 5 (Drosophila) |
| gene expression | Tbx5 | T-box 5 |
| gene expression | Nppa | Natriuretic peptide type A |
| gene expression | Acta1 | Actin, alpha 1, skeletal muscle |
| gene expression | Adra1b | Adrenergic receptor, alpha 1b |
| gene expression | Adra2a | Adrenergic receptor, alpha 2a |
| gene expression | Actc1 | Actin, alpha, cardiac muscle 1 |
| gene expression | Actn1 | Actinin, alpha 1 |
| gene expression | Actn2 | Actinin alpha 2 |
| gene expression | Pln | Phospholamban |
| gene expression | Tnnt2 | Troponin T2, cardiac |
| gene expression | Ttn | Titin |
| gene expression | Myh6 | Myosin, heavy polypeptide 6, cardiac muscle, alpha |
| gene expression | Myh7 | Myosin, heavy polypeptide 7, cardiac muscle, beta |
| gene expression | Myl2 | Myosin, light polypeptide 2, regulatory, cardiac, slow |
| gene expression | Myl3 | Myosin, light polypeptide 3 |
| gene expression | Myl4 | Myosin, light polypeptide 4 |
| gene expression | Myl7 | Myosin, light polypeptide 7, regulatory |
| gene expression | Cacna1c | Calcium channel, voltage-dependent, L type, alpha 1C subunit |
| gene expression | Cacna1d | Calcium channel, voltage-dependent, L type, alpha 1D subunit |
| gene expression | Cacna1g | Calcium channel, voltage-dependent, T type, alpha 1G subunit |
| gene expression | Cacna1h | Calcium channel, voltage-dependent, T type, alpha 1H subunit |
| gene expression | Kcne1 | Potassium voltage-gated channel, Isk-related subfamily, member 1 |
| gene expression | Kcne2 | Potassium voltage-gated channel, Isk-related subfamily, gene 2 |
| gene expression | Kcnd2 | Potassium voltage-gated channel, Shal-related family, member 2 |
| gene expression | Kcnd3 | Potassium voltage-gated channel, Shal-related family, member 3 |
| gene expression | Kcnh2 | Potassium voltage-gated channel, subfamily H (eag-related), member 2 |
| gene expression | Kcnj2 | Potassium inwardly-rectifying channel, subfamily J, member 2 |
| gene expression | Kcnj3 | Potassium inwardly-rectifying channel, subfamily J, member 3 |
| gene expression | Kcnj11 | Potassium inwardly rectifying channel, subfamily J, member 11 |
| gene expression | Kcnj12 | Potassium inwardly-rectifying channel, subfamily J, member 12 |
| gene expression | Kcnj14 | Potassium inwardly-rectifying channel, subfamily J, member 14 |
| gene expression | Kcnq1 | Potassium voltage-gated channel, subfamily Q, member 1 |
| gene expression | Scn5a | Sodium channel, voltage-gated, type V, alpha |
| gene expression | Slc2a1 | Solute carrier family 2 (facilitated glucose transporter), member 1 |
| gene expression | Slc2a2 | Solute carrier family 2 (facilitated glucose transporter), member 2 |
| gene expression | Slc8a1 | Solute carrier family 8 (sodium/calcium exchanger), member 1 |
| gene expression | Hcn1 | Hyperpolarization-activated, cyclic nucleotide-gated K+ 1 |
| gene expression | Hcn3 | Hyperpolarization-activated, cyclic nucleotide-gated K+ 3 |
| gene expression | Hcn4 | Hyperpolarization-activated, cyclic nucleotide-gated K+ 4 |
| gene expression | Gja1 | Gap junction protein, alpha 1 |
| gene expression | Gja5 | Gap junction protein, alpha 5 |
| gene expression | Atp1a2 | ATPase, Ca++ transporting, cardiac muscle, fast twitch 1 |
| gene expression | Atp2a2 | ATPase, Ca++ transporting, cardiac muscle, slow twitch 2 |
| gene expression | Ryr2 | Ryanodine receptor 2, cardiac |
| gene expression | Ckm | Creatine kinase, muscle |

TABLE 3
Custom RT-qPCR array gene list

| Gene Symbol | Refseq # | Gene Description |
|---|---|---|
| Hey2 | NM_013904 | Hairy/enhancer-of-split related with YRPW motif 2 |
| Irx4 | NM_018885 | Iroquois related homeobox 4 (Drosophila) |
| Bmp10 | NM_009756 | Bone morphogenetic protein 10 |
| Gata4 | NM_008092 | GATA binding protein 4 |
| Myocd | NM_145136 | Myocardin |
| Nkx2-5 | NM_008700 | NK2 transcription factor related, locus 5 (Drosophila) |
| Tbx5 | NM_011537 | T-box 5 |
| Nppa | NM_008725 | Natriuretic peptide type A |
| Acta1 | NM_009606 | Actin, alpha 1, skeletal muscle |
| Adra1b | NM_007416 | Adrenergic receptor, alpha 1b |
| Adra2a | NM_007417 | Adrenergic receptor, alpha 2a |
| Actc1 | NM_009608 | Actin, alpha, cardiac muscle 1 |
| Actn1 | NM_134156 | Actinin, alpha 1 |
| Actn2 | NM_033268 | Actinin alpha 2 |
| Pln | NM_023129 | Phospholamban |
| Tnnt2 | NM_011619 | Troponin T2, cardiac |
| Ttn | NM_011652 | Titin |
| Myh6 | NM_010856 | Myosin, heavy polypeptide 6, cardiac muscle, alpha |
| Myh7 | NM_080728 | Myosin, heavy polypeptide 7, cardiac muscle, beta |
| Myl2 | NM_010861 | Myosin, light polypeptide 2, regulatory, cardiac, slow |
| Myl3 | NM_010859 | Myosin, light polypeptide 3 |
| Myl4 | NM_010858 | Myosin, light polypeptide 4 |
| Myl7 | NM_022879 | Myosin, light polypeptide 7, regulatory |
| Cacna1c | NM_009781 | Calcium channel, voltage-dependent, L type, alpha 1C subunit |
| Cacna1d | NM_028981 | Calcium channel, voltage-dependent, L type, alpha 1D subunit |
| Cacna1g | NM_009783 | Calcium channel, voltage-dependent, T type, alpha 1G subunit |
| Cacna1h | NM_021415 | Calcium channel, voltage-dependent, T type, alpha 1H subunit |
| Kcna5 | NM_145983 | Potassium voltage-gated channel, shaker-related subfamily, member 5 |
| Kcne1 | NM_008424 | Potassium voltage-gated channel, Isk-related subfamily, member 1 |
| Kcne2 | NM_134110 | Potassium voltage-gated channel, Isk-related subfamily, gene 2 |
| Kcnd2 | NM_019697 | Potassium voltage-gated channel, Shal-related family, member 2 |
| Kcnd3 | NM_019931 | Potassium voltage-gated channel, Shal-related family, member 3 |
| Kcnh2 | NM_013569 | Potassium voltage-gated channel, subfamily H (eag-related), member 2 |
| Kcnj2 | NM_008425 | Potassium inwardly-rectifying channel, subfamily J, member 2 |
| Kcnj3 | NM_008426 | Potassium inwardly-rectifying channel, subfamily J, member 3 |
| Kcnj11 | NM_010602 | Potassium inwardly rectifying channel, subfamily J, member 11 |
| Kcnj12 | NM_010603 | Potassium inwardly-rectifying channel, subfamily J, member 12 |
| Kcnj14 | NM_145963 | Potassium inwardly-rectifying channel, subfamily J, member 14 |
| Kcnq1 | NM_008434 | Potassium voltage-gated channel, subfamily Q, member 1 |
| Scn5a | NM_021544 | Sodium channel, voltage-gated, type V, alpha |
| Slc2a1 | NM_011400 | Solute carrier family 2 (facilitated glucose transporter), member 1 |
| Slc2a2 | NM_031197 | Solute carrier family 2 (facilitated glucose transporter), member 2 |
| Slc8a1 | NM_011406 | Solute carrier family 8 (sodium/calcium exchanger), member 1 |
| Hcn1 | NM_010408 | Hyperpolarization-activated, cyclic nucleotide-gated K+ 1 |
| Hcn3 | NM_008227 | Hyperpolarization-activated, cyclic nucleotide-gated K+ 3 |
| Hcn4 | NM_001081192 | Hyperpolarization-activated, cyclic nucleotide-gated K+ 4 |
| Gja1 | NM_010288 | Gap junction protein, alpha 1 |
| Gja5 | NM_008121 | Gap junction protein, alpha 5 |
| Atp1a2 | NM_178405 | ATPase, Na+/K+ transporting, alpha 2 polypeptide |
| Atp1a3 | NM_144921 | ATPase, Na+/K+ transporting, alpha 3 polypeptide |
| Atp2a1 | NM_007504 | ATPase, Ca++ transporting, cardiac muscle, fast twitch 1 |
| Atp2a2 | NM_009722 | ATPase, Ca++ transporting, cardiac muscle, slow twitch 2 |
| Ryr2 | NM_023868 | Ryanodine receptor 2, cardiac |
| Ckm | NM_007710 | Creatine kinase, muscle |
| Acsl5 | NM_027976 | Acyl-CoA synthetase long-chain family member 5 |
| Ptk2 | NM_007982 | PTK2 protein tyrosine kinase 2 |
| Ilk | NM_010562 | Integrin linked kinase |
| Ctgf | NM_010217 | Connective tissue growth factor |
| Itga1 | NM_001033228 | Integrin alpha 1 |
| Itga2 | NM_008396 | Integrin alpha 2 |
| Itga4 | NM_010576 | Integrin alpha 4 |
| Itga5 | NM_010577 | Integrin alpha 5 (fibronectin receptor alpha) |
| Itgav | NM_008402 | Integrin alpha V |
| Itgb1 | NM_010578 | Integrin beta 1 (fibronectin receptor beta) |
| Itgb3 | NM_016780 | Integrin beta 3 |
| Abra | NM_175456 | Actin-binding Rho activating protein |
| Rhoa | NM_016802 | Ras homolog gene family, member A |
| Cdc42 | NM_009861 | Cell division cycle 42 homolog (S. cerevisiae) |
| Rac1 | NM_009007 | RAS-related C3 botulinum substrate 1 |
| Rock1 | NM_009071 | Rho-associated coiled-coil containing protein kinase 1 |
| Rock2 | NM_009072 | Rho-associated coiled-coil containing protein kinase 2 |
| Rnd1 | NM_172612 | Rho family GTPase 1 |
| Vcl | NM_009502 | Vinculin |
| Ctnnb1 | NM_007614 | Catenin (cadherin associated protein), beta 1 |
| Aifm1 | NM_012019 | Apoptosis-inducing factor, mitochondrion-associated 1 |
| Atp5j | NM_016755 | ATP synthase, H+ transporting, mitochondrial F0 complex, subunit F |
| Hsp90ab1 | NM_008302 | Heat shock protein 90 alpha (cytosolic), class B member 1 |
| Hspa2 | NM_008301 | Heat shock protein 2 |
| Hsph1 | NM_013559 | Heat shock 105 kDa/110 kDa protein 1 |
| Bcat1 | NM_007532 | Branched chain aminotransferase 1, cytosolic |
| Ch25h | NM_009890 | Cholesterol 25-hydroxylase |
| Itpr2 | NM_019923 | Inositol 1,4,5-triphosphate receptor 2 |
| Tgfb2 | NM_009367 | Transforming growth factor, beta 2 |
| Notch1 | NM_008714 | Notch gene homolog 1 (Drosophila) |
| Pou5f1 | NM_013633 | POU domain, class 5, transcription factor 1 |
| Nanog | NM_028016 | Nanog homeobox |
| Sox2 | NM_011443 | SRY-box containing gene 2 |
| Gapdh | NM_008084 | Glyceraldehyde-3-phosphate dehydrogenase |
| Actb | NM_007393 | Actin, beta |

TABLE 4

| Measurement | Measure- | NMVM | NMVM | | mES | | miPS |
|---|---|---|---|---|---|---|---|
| Class | ment | Mean | Std. Dev. | mES Mean | Std. Dev. | miPS Mean | Std. Dev. |
| contractility | Diastolic | 7.5803 | 1.466033574 | 2.66413125 | 0.851402982 | 4.169469231 | 2.141664999 |
| contractility | Systolic | 16.78524545 | 4.991782588 | 3.06475625 | 0.952736187 | 5.221469231 | 2.402833691 |
| contractility | Twitch | 9.204945455 | 4.850426303 | 0.41495 | 0.284532454 | 1.051992308 | 0.583580089 |
| electrophysiology | LCV | 22.81400669 | 4.405245591 | 20.18802529 | 4.067156955 | 16.80914176 | 3.0744218 |
| electrophysiology | TCV | 8.274104129 | 2.97538821 | 4.434581496 | 1.570138306 | 5.897009011 | 2.676070439 |
| electrophysiology | AR | 3.281458159 | 1.630927247 | 5.156401256 | 2.035955393 | 3.2629691 | 1.013367736 |
| electrophysiology | APD50 | 0.0602 | 0.021018087 | 0.0216 | 0.004963869 | 0.0404 | 0.005122499 |
| electrophysiology | APD90 | 0.1672 | 0.030792207 | 0.103 | 0.025353501 | 0.12 | 0.026974062 |
| electrophysiology | TOT | −104.842 | 69 | −48.175 | 27.225 | −61.905 | 32.832 |
| electrophysiology | LCC | −45.327 | 45.642 | −24.505 | 17.793 | −23.185 | 20.517 |
| electrophysiology | TCC | −76.49 | 49.184 | −29.4 | 22.679 | −45.935 | 32.165 |
| morphology | SPD | 0.2245375 | 0.053934577 | 0.158088636 | 0.052702002 | 0.124964444 | 0.036314167 |
| morphology | SL | 1.919315909 | 0.235940133 | 1.776267568 | 0.113561051 | 1.728273973 | 0.149846282 |
| morphology | OOP | 0.71985 | 0.121625868 | 0.358075862 | 0.177490728 | 0.439355556 | 0.183381891 |
| gene expression | Hey2 | −9.770145959 | 0.573320531 | −10.54235623 | 0.366640689 | −11.11822775 | 0.826189804 |
| gene expression | Irx4 | −6.625040753 | 0.609298159 | −9.600002357 | 0.769993321 | −7.138810122 | 0.347093688 |
| gene expression | Gata4 | −8.517075533 | 0.623732452 | −7.674579895 | 0.631473135 | −8.174181439 | 0.472243496 |
| gene expression | Myocd | −5.532772375 | 0.479609122 | −4.501276702 | 0.456034122 | −4.431552241 | 0.660442543 |
| gene expression | Nkx2-5 | −12.99047239 | 3.33521095 | −10.0596879 | 1.630913947 | −10.68227401 | 0.771923063 |
| gene expression | Tbx5 | −10.88196276 | 1.168528463 | −6.897520263 | 0.480848245 | −8.661808742 | 0.481015744 |
| gene expression | Nppa | 2.99495338 | 0.611152769 | −0.838055065 | 0.025398127 | −2.082299676 | 0.106044236 |
| gene expression | Acta1 | 0.1755231 | 0.734129068 | −4.363305147 | 1.02089301 | −3.29288943 | 0.759496029 |
| gene expression | Adra1b | −7.470203758 | 1.174075334 | −6.673218328 | 0.095460622 | −7.918226613 | 0.148713683 |
| gene expression | Adra2a | −16.06224899 | 1.438842435 | −13.50917129 | 0.738202701 | −15.11406134 | 0.342905184 |
| gene expression | Actc1 | 0.070300809 | 0.369136956 | 2.427621724 | 0.445702907 | 2.515407327 | 0.739160974 |
| gene expression | Actn1 | −7.312518401 | 1.604283769 | −6.560366641 | 0.786844552 | −6.926768645 | 0.337289238 |
| gene expression | Actn2 | −1.73137147 | 0.871025214 | −1.220110687 | 0.224666693 | −1.769354955 | 0.524499442 |
| gene expression | Pln | −2.072155636 | 0.265620597 | −5.52514239 | 0.319448372 | −3.988544942 | 0.639026121 |
| gene expression | Tnnt2 | 1.135898492 | 0.513342055 | 1.41684189 | 0.573266073 | 1.561329224 | 0.421071301 |
| gene expression | Ttn | −1.607340655 | 0.694088066 | −1.61045564 | 0.595743754 | −1.978802767 | 0.118577912 |
| gene expression | Myh6 | 1.79488808 | 1.419803007 | 1.567753424 | 0.559601589 | 0.94271039 | 0.204921569 |
| gene expression | Myh7 | −1.849579046 | 0.605911976 | −1.179091853 | 0.053634897 | 1.184581558 | 0.363838496 |
| gene expression | Myl2 | 1.115869917 | 0.687795975 | 0.07977807 | 0.303475325 | 0.991003722 | 0.504396011 |
| gene expression | Myl3 | 0.634682134 | 0.627269278 | −0.443794232 | 0.285062612 | 0.382814953 | 0.635627428 |
| gene expression | Myl4 | −6.660350265 | 1.31841278 | 1.333058987 | 0.353672251 | 0.060616713 | 0.318095365 |
| gene expression | Myl7 | −4.785320077 | 2.725187597 | 1.915489621 | 0.410293466 | 0.666778924 | 1.654436722 |
| gene expression | Cacna1c | −6.631864611 | 0.649347999 | −6.185466635 | 0.596928023 | −6.04354066 | 0.215318457 |
| gene expression | Cacna1d | −18.0700418 | 2.669683546 | −12.65496936 | 0.046715387 | −12.67893045 | 0.751276131 |
| gene expression | Cacna1g | −10.73155023 | 1.558665015 | −6.394148401 | 0.528025899 | −7.998959442 | 0.704656647 |
| gene expression | Cacna1h | −11.49362726 | 2.182349747 | −6.922184923 | 0.612356632 | −8.306623576 | 1.851522486 |
| gene expression | Kcne1 | −8.432061674 | 0.666305364 | −10.39141046 | 0.691608228 | −9.632546046 | 0.314421481 |
| gene expression | Kcne2 | −15.66255553 | 1.14616704 | −13.51523982 | 0.224721171 | −13.19809545 | 0.579382687 |
| gene expression | Kcnd2 | −10.17497848 | 0.609310929 | −12.50846509 | 0.184708689 | −13.50847849 | 0.839600341 |
| gene expression | Kcnd3 | −12.893414 | 0.791739907 | −12.51273079 | 0.236690053 | −13.09615191 | 0.416040456 |
| gene expression | Kcnh2 | −11.67957059 | 3.878256882 | −7.724266578 | 0.413806742 | −9.787438451 | 2.113002486 |
| gene expression | Kcnj2 | −9.534715964 | 1.899378523 | −15.089209 | 2.061084187 | −14.59505077 | 1.594688381 |
| gene expression | Kcnj3 | −14.89341398 | 2.00798793 | −11.83382256 | 1.391114349 | −14.75961064 | 0.352044869 |
| gene expression | Kcnj11 | −12.57473906 | 1.631489124 | −11.91485585 | 1.486298461 | −12.16151676 | 0.953235691 |
| gene expression | Kcnj12 | −10.93743907 | 1.657743837 | −11.67031353 | 1.324626819 | −12.50347414 | 0.504980619 |
| gene expression | Kcnj14 | −13.84781047 | 1.918846507 | −16.00563692 | 0.549772231 | −20.54864593 | 5.771404952 |
| gene expression | Kcnq1 | −8.665375372 | 1.242585062 | −10.98252451 | 0.603999105 | −13.38251396 | 0.318575481 |
| gene expression | Scn5a | −6.276982192 | 2.010587216 | −7.032992975 | 0.219600465 | −8.555893139 | 0.59030839 |
| gene expression | Slc2a1 | −5.5100847 | 0.736079521 | −4.537434845 | 0.130370029 | −4.687040538 | 0.311971825 |
| gene expression | Slc2a2 | −16.19021672 | 1.145891593 | −15.5444213 | 0.86598448 | −16.52039322 | 0.56634507 |
| gene expression | Slc8a1 | −3.752917995 | 0.84787214 | −2.716713418 | 0.474665842 | −3.741171284 | 0.130382016 |
| gene expression | Hcn1 | −9.595017657 | 1.096559766 | −7.709111124 | 0.481128276 | −7.656291492 | 0.143019895 |
| gene expression | Hcn3 | −16.58516194 | 1.045115504 | −15.47507572 | 0.134226966 | −17.69138643 | 0.275566231 |
| gene expression | Hcn4 | −15.51944661 | 2.329368729 | −8.720813883 | 0.484968241 | −9.291547797 | 0.580891093 |
| gene expression | Gja1 | −5.124187088 | 2.21013532 | −7.226294085 | 1.202823121 | −7.341755142 | 1.559297987 |
| gene expression | Gja5 | −15.34410991 | 2.701673604 | −15.92011063 | 2.359653092 | −18.13090691 | 0.847740178 |
| gene expression | Atp1a2 | −7.730754383 | 0.576131949 | −6.123154697 | 0.167951234 | −6.968143377 | 0.444394468 |
| gene expression | Atp2a2 | −0.261609648 | 0.690175243 | 0.530402188 | 0.377534065 | 0.064256217 | 0.759050017 |
| gene expression | Ryr2 | −2.293483998 | 0.720008911 | −2.90050403 | 0.282140961 | −2.961976782 | 0.221891581 |
| gene expression | Ckm | −6.044739072 | 3.170959922 | −9.72194035 | 0.730625102 | −9.331677833 | 1.255731727 |

| | | | Norm. | Norm. | |
|---|---|---|---|---|---|
| Measurement | Min Class | Max Class | NMVM | NMVM | Norm. mES |
| Class | Mean | Mean | Mean | St. Dev. | Mean |
| contractility | 0.41495 | 16.78524545 | 0.437704379 | 0.089554497 | 0.137394054 |
| contractility | 0.41495 | 16.78524545 | 1 | 0.304929291 | 0.161866733 |
| contractility | 0.41495 | 16.78524545 | 0.536947881 | 0.296294365 | 0 |
| electrophysiology | 3.2629691 | 22.81400669 | 1 | 0.225320297 | 0.865685829 |
| electrophysiology | 3.2629691 | 22.81400669 | 0.256310439 | 0.152185693 | 0.059925842 |
| electrophysiology | 3.2629691 | 22.81400669 | 0.000945682 | 0.083418961 | 0.09684561 |
| electrophysiology | 0.0216 | 0.1672 | 0.26510989 | 0.144354993 | 0 |
| electrophysiology | 0.0216 | 0.1672 | 1 | 0.211484938 | 0.559065934 |
| electrophysiology | −104.842 | −23.185 | 0 | 0.844997979 | 0.693963775 |
| electrophysiology | −104.842 | −23.185 | 0.728841373 | 0.558947794 | 0.983834821 |
| electrophysiology | −104.842 | −23.185 | 0.347208445 | 0.602324357 | 0.92388895 |
| morphology | 0.124964444 | 1.919315909 | 0.055492504 | 0.030057978 | 0.018460259 |
| morphology | 0.124964444 | 1.919315909 | 1 | 0.131490479 | 0.920278527 |
| morphology | 0.124964444 | 1.919315909 | 0.331532349 | 0.067782634 | 0.129914023 |
| gene expression | −20.54864593 | 2.99495338 | 0.457810203 | 0.024351439 | 0.425011043 |
| gene expression | −20.54864593 | 2.99495338 | 0.591396625 | 0.025879567 | 0.465036948 |
| gene expression | −20.54864593 | 2.99495338 | 0.511033604 | 0.026492655 | 0.546818091 |
| gene expression | −20.54864593 | 2.99495338 | 0.637790057 | 0.020371105 | 0.681602206 |
| gene expression | −20.54864593 | 2.99495338 | 0.321028805 | 0.141661048 | 0.445512086 |
| gene expression | −20.54864593 | 2.99495338 | 0.410586463 | 0.049632533 | 0.579823225 |
| gene expression | −20.54864593 | 2.99495338 | 1 | 0.025958341 | 0.837195308 |
| gene expression | −20.54864593 | 2.99495338 | 0.880246421 | 0.031181684 | 0.687462464 |
| gene expression | −20.54864593 | 2.99495338 | 0.555498843 | 0.049868133 | 0.589350312 |
| gene expression | −20.54864593 | 2.99495338 | 0.19055697 | 0.061113954 | 0.298997385 |
| gene expression | −20.54864593 | 2.99495338 | 0.875777168 | 0.015678867 | 0.975902934 |
| gene expression | −20.54864593 | 2.99495338 | 0.562196432 | 0.068140973 | 0.594143619 |
| gene expression | −20.54864593 | 2.99495338 | 0.799252239 | 0.036996264 | 0.820967728 |
| gene expression | −20.54864593 | 2.99495338 | 0.78477764 | 0.011282073 | 0.638114136 |
| gene expression | −20.54864593 | 2.99495338 | 0.921037779 | 0.02180389 | 0.932970678 |
| gene expression | −20.54864593 | 2.99495338 | 0.804520372 | 0.029480967 | 0.804388065 |
| gene expression | −20.54864593 | 2.99495338 | 0.949027959 | 0.060305265 | 0.939380554 |
| gene expression | −20.54864593 | 2.99495338 | 0.794231444 | 0.025735741 | 0.822709978 |
| gene expression | −20.54864593 | 2.99495338 | 0.920187078 | 0.029213714 | 0.876179709 |
| gene expression | −20.54864593 | 2.99495338 | 0.89974892 | 0.026642879 | 0.853941296 |
| gene expression | −20.54864593 | 2.99495338 | 0.589896875 | 0.055998778 | 0.929412051 |
| gene expression | −20.54864593 | 2.99495338 | 0.669537637 | 0.115750679 | 0.954150436 |
| gene expression | −20.54864593 | 2.99495338 | 0.591106786 | 0.02758066 | 0.610067267 |
| gene expression | −20.54864593 | 2.99495338 | 0.105277197 | 0.113393178 | 0.335279091 |
| gene expression | −20.54864593 | 2.99495338 | 0.416975143 | 0.066203344 | 0.601203637 |
| gene expression | −20.54864593 | 2.99495338 | 0.384606387 | 0.092693972 | 0.578775608 |
| gene expression | −20.54864593 | 2.99495338 | 0.514644515 | 0.028300913 | 0.431422373 |
| gene expression | −20.54864593 | 2.99495338 | 0.207533705 | 0.048682745 | 0.298739628 |
| gene expression | −20.54864593 | 2.99495338 | 0.440615189 | 0.02588011 | 0.34150177 |
| gene expression | −20.54864593 | 2.99495338 | 0.325151301 | 0.033628669 | 0.341320587 |
| gene expression | −20.54864593 | 2.99495338 | 0.376708558 | 0.164726592 | 0.54470768 |
| gene expression | −20.54864593 | 2.99495338 | 0.467809948 | 0.080674943 | 0.231886249 |
| gene expression | −20.54864593 | 2.99495338 | 0.240202523 | 0.085288061 | 0.3701568 |
| gene expression | −20.54864593 | 2.99495338 | 0.338686824 | 0.069296504 | 0.36671496 |
| gene expression | −20.54864593 | 2.99495338 | 0.408230141 | 0.070411657 | 0.377101746 |
| gene expression | −20.54864593 | 2.99495338 | 0.284613893 | 0.081501833 | 0.192961533 |
| gene expression | −20.54864593 | 2.99495338 | 0.504734659 | 0.052778042 | 0.406315164 |
| gene expression | −20.54864593 | 2.99495338 | 0.6061802 | 0.085398464 | 0.574069104 |
| gene expression | −20.54864593 | 2.99495338 | 0.638753703 | 0.031264528 | 0.680066411 |
| gene expression | −20.54864593 | 2.99495338 | 0.185121619 | 0.048671045 | 0.212551385 |
| gene expression | −20.54864593 | 2.99495338 | 0.713388285 | 0.036012851 | 0.757400442 |
| gene expression | −20.54864593 | 2.99495338 | 0.46524867 | 0.046575706 | 0.545351398 |
| gene expression | −20.54864593 | 2.99495338 | 0.168346562 | 0.044390643 | 0.215496796 |
| gene expression | −20.54864593 | 2.99495338 | 0.213612169 | 0.098938514 | 0.502379942 |
| gene expression | −20.54864593 | 2.99495338 | 0.655144468 | 0.093874148 | 0.565858757 |
| gene expression | −20.54864593 | 2.99495338 | 0.221059489 | 0.114751936 | 0.19659421 |
| gene expression | −20.54864593 | 2.99495338 | 0.544432114 | 0.024470853 | 0.612713929 |
| gene expression | −20.54864593 | 2.99495338 | 0.861679475 | 0.029314772 | 0.895319693 |
| gene expression | −20.54864593 | 2.99495338 | 0.775376853 | 0.030581939 | 0.749594048 |
| gene expression | −20.54864593 | 2.99495338 | 0.616044585 | 0.134684586 | 0.459857706 |

| | | | | NMVM vs. | NMVM vs. |
|---|---|---|---|---|---|
| Measurement | Norm mES | Norm. miPS | Norm. miPS | mES SSMD | miPS SSMD |
| Class | Std. Dev. | Mean | Std. Dev. | (β mES) | (β miPS) |
| contractility | 0.052009017 | 0.229349509 | 0.130826289 | −2.89983115 | −1.314194568 |
| contractility | 0.058199083 | 0.293612247 | 0.146780105 | −2.699879549 | −2.087327521 |
| contractility | 0.017381021 | 0.038914527 | 0.035648721 | −1.809100886 | −1.668838079 |
| electrophysiology | 0.208027678 | 0.692862085 | 0.157251081 | −0.437980278 | −1.117810504 |
| electrophysiology | 0.080309718 | 0.134726349 | 0.136876134 | −1.141266897 | −0.594008614 |
| electrophysiology | 0.104135414 | 0 | 0.051831916 | 0.718741651 | −0.009629146 |
| electrophysiology | 0.034092507 | 0.129120879 | 0.035181999 | −1.78734358 | −0.915255346 |
| electrophysiology | 0.174131188 | 0.675824176 | 0.185261415 | −1.609553417 | −1.153017993 |
| electrophysiology | 0.333406811 | 0.525821424 | 0.402072082 | 0.763944855 | 0.561907311 |
| electrophysiology | 0.217899262 | 1 | 0.251258312 | 0.42504651 | 0.442473832 |
| electrophysiology | 0.277734916 | 0.721395594 | 0.393903768 | 0.869446437 | 0.519927672 |
| morphology | 0.029371059 | 0 | 0.020238046 | −0.881185506 | −1.531411653 |
| morphology | 0.063288076 | 0.893531485 | 0.083509995 | −0.546305031 | −0.683507043 |
| morphology | 0.098916367 | 0.175211556 | 0.102199538 | −1.681383079 | −1.274688108 |
| gene expression | 0.015572839 | 0.400551252 | 0.035091907 | −1.134718017 | −1.340538344 |
| gene expression | 0.032704996 | 0.569574585 | 0.014742592 | −3.029791023 | −0.73267274 |
| gene expression | 0.026821436 | 0.525597821 | 0.020058254 | 0.949203952 | 0.438292857 |
| gene expression | 0.019369771 | 0.68456371 | 0.028051894 | 1.558598688 | 1.34917663 |
| gene expression | 0.069272074 | 0.419068121 | 0.032786961 | 0.789412381 | 0.674246405 |
| gene expression | 0.020423736 | 0.504886149 | 0.02043085 | 3.15325769 | 1.756924478 |
| gene expression | 0.00107877 | 0.784346778 | 0.004504164 | −6.266359298 | −8.185359403 |
| gene expression | 0.043361807 | 0.732927717 | 0.03225913 | −3.609563923 | −3.283533998 |
| gene expression | 0.004054632 | 0.536469346 | 0.006316523 | 0.67658695 | −0.378571546 |
| gene expression | 0.031354709 | 0.230830661 | 0.014564688 | 1.578740251 | 0.641040357 |
| gene expression | 0.018930959 | 0.979631574 | 0.031395411 | 4.073358123 | 2.959429161 |
| gene expression | 0.033420742 | 0.578580917 | 0.014326154 | 0.420936094 | 0.235305564 |
| gene expression | 0.009542581 | 0.797638914 | 0.022277793 | 0.568362304 | −0.037357669 |
| gene expression | 0.013568374 | 0.703380174 | 0.027142244 | −8.311368714 | −2.769219507 |
| gene expression | 0.024349126 | 0.93910769 | 0.017884746 | 0.365091293 | 0.640763358 |
| gene expression | 0.025303852 | 0.788742746 | 0.005036524 | −0.003405487 | −0.527537038 |
| gene expression | 0.023768736 | 0.912832233 | 0.008703918 | −0.148832978 | −0.594052806 |
| gene expression | 0.002278109 | 0.923105563 | 0.015453818 | 1.102265181 | 4.293064757 |
| gene expression | 0.012889929 | 0.914883462 | 0.021423912 | −1.378200258 | −0.146397869 |
| gene expression | 0.01210786 | 0.889051016 | 0.026997887 | −1.565267127 | −0.282039046 |
| gene expression | 0.015022013 | 0.875365842 | 0.013510906 | 5.855865403 | 4.955574782 |
| gene expression | 0.017426964 | 0.901112212 | 0.070271189 | 2.431440988 | 1.710155103 |
| gene expression | 0.025354153 | 0.616095487 | 0.00914552 | 0.506103275 | 0.859976744 |
| gene expression | 0.001984208 | 0.334261358 | 0.031909995 | 2.028046957 | 1.943878967 |
| gene expression | 0.022427578 | 0.533040268 | 0.029929861 | 2.635636083 | 1.597493502 |
| gene expression | 0.026009474 | 0.519972422 | 0.078642287 | 2.016841594 | 1.113575784 |
| gene expression | 0.029375637 | 0.463654675 | 0.01335486 | −2.040230476 | −1.629397735 |
| gene expression | 0.009544894 | 0.312210142 | 0.024608926 | 1.83847242 | 1.91893828 |
| gene expression | 0.007845389 | 0.299026812 | 0.035661512 | −3.665014544 | −3.213337032 |
| gene expression | 0.010053265 | 0.316540132 | 0.017671064 | 0.460673624 | −0.22667613 |
| gene expression | 0.017576189 | 0.457075715 | 0.08974849 | 1.014110096 | 0.428421427 |
| gene expression | 0.087543292 | 0.252875318 | 0.067733415 | −1.981763356 | −2.040412828 |
| gene expression | 0.059086732 | 0.245885738 | 0.014952891 | 1.252499623 | 0.065634433 |
| gene expression | 0.063129619 | 0.356238189 | 0.040488104 | 0.298995843 | 0.218687723 |
| gene expression | 0.056262715 | 0.341713758 | 0.021448743 | −0.345374786 | −0.903680967 |
| gene expression | 0.02335124 | 0 | 0.2451369 | −1.081047504 | −1.101743272 |
| gene expression | 0.025654493 | 0.304377079 | 0.013531299 | −1.67714291 | −3.677296427 |
| gene expression | 0.009327396 | 0.50938485 | 0.025072988 | −0.373791959 | −1.087550288 |
| gene expression | 0.005537387 | 0.673712001 | 0.013250813 | 1.301141931 | 1.029497547 |
| gene expression | 0.036782162 | 0.171097573 | 0.024055161 | 0.449620081 | −0.258312143 |
| gene expression | 0.020161142 | 0.713887219 | 0.005537896 | 1.066386646 | 0.013693386 |
| gene expression | 0.02043563 | 0.54759488 | 0.006074683 | 1.57491287 | 1.753159189 |
| gene expression | 0.005701208 | 0.121360352 | 0.011704507 | 1.053512783 | −1.02349112 |
| gene expression | 0.02059873 | 0.478138367 | 0.024672994 | 2.857387492 | 2.59419385 |
| gene expression | 0.051089177 | 0.560954619 | 0.06623023 | −0.835414759 | −0.819854703 |
| gene expression | 0.100224824 | 0.102691988 | 0.036007246 | −0.160577365 | −0.984193142 |
| gene expression | 0.007133626 | 0.576823551 | 0.018875384 | 2.678828195 | 1.048105901 |
| gene expression | 0.016035529 | 0.875520428 | 0.032240186 | 1.006770799 | 0.317634792 |
| gene expression | 0.011983765 | 0.74698303 | 0.009424709 | −0.784958132 | −0.887272248 |
| gene expression | 0.031032855 | 0.476433869 | 0.053336438 | −1.130040562 | −0.963756311 |

| | | |
|---|---|---|
| Stem Cell Quality Index | 4.951758974 | 3.598649069 |

### Example 2

### Metrics of Cytoskeletal Organization to Identify the Structural Phenotypes of Stem Cell Derived Cardiomyocytes

As demonstrated in herein, human induced pluripotent stem cell derived myocytes exhibited qualitatively and quantitatively underdeveloped contractile cytoskeletons with respect to murine primary and stem cell derived cardiomyocytes when exposed to in-vivo like experimental conditions. This is consistent with the notion that human stem cell derived cardiomyocytes may require longer time in culture or ad-hoc conditioning to fully mature, and suggests that metrics of cytoskeleton architecture can be utilized to quantitatively monitor this process. Accordingly, in addition to the metric parameters described in Example 1, a new metric of cytoskeletal organization, the sarcomere packing density, has been developed to further distinguish architectural phenotypes in establishment of the quality index used in the system and method of the present invention.

Demonstrated herein is a novel metric, the sarcomere packing density, that quantifies the presence of fully formed sarcomeres and provides an estimate for the maturity of the contractile cytoskeleton. The question was asked whether this metric could be utilized to perform structural phenotyping of stem cell derived cardiomyocytes. To answer this question immunocytochemistry analysis was performed of the cell cytoskeleton in primary (neonate mouse) and commercially available human and murine induced pluripotent stem cell derived cardiomyocytes cultured on engineered substrates that recapitulate the chemo-mechanical properties of the native microenvironment (McCain et al., 2012, Proc Natl Acad Sci USA 109:9881-9886). The experiments of Example 2 revealed that the sarcomere packing density numerically quantifies the inability of human induced pluripotent stem cell derived cardiomyocytes to assemble the kind of contractile cytoskeleton observed in murine primary and stem cell derived cardiomyocytes under the same experimental conditions.

The following materials and methods were used in Example 2. In brief, cell suspensions of primary cardiomyocytes (pCMs) were directly obtained from primary neonate mouse harvest while cultures of human (iCells from Cellular Dynamics International, Madison, Wis.) and murine (CorAt from Axiogenesis, Cologne, Germany) induced pluripotent stem cell derived cardiomyocytes (respectively hiCMs and miCMs) were obtained following the manufacturers' guidance.

All cell types were seeded on polyacrylamide gels engineered (McCain et al., 2012, Proc Natl Acad Sci USA 109:9881-9886) to a nominal substrate stiffness of 13 kPa and decorated with micro-contact printed fibronectin islands (BD Biosciences, Bedford, Mass.). Cells were cultured on the substrates with regular media exchanges for 72 hour and subsequently fixed and stained with primary antibodies: Alexa633-phalloidin (A22284 Invitrogen), DAPI (D3571 Invitrogen), anti-mouse sarcomeric α-actinin (A881 Sigma) and anti-human fibronectin (F3648 Sigma); and secondary antibodies: GAM-alexa546 (A21143 Invitrogen) and GAR-alexa488 (A11008 Invitrogen). Mono-nucleated, fully spread single cells were imaged with a confocal line scanning microscope (Zeiss LSM510 live). Micrographs were preprocessed in FIJI (Schindelin et al., 2012, Nature Methods 9:676-682) to detect filamentous cytoskeletal structures (Sato et al., 1998, Medical Image Analysis 2: 143-168) and their orientations (Rezakhaniha et al., 2011, Biomech Model Mechanobiol 11:461-473). Finally, Matlab (Mathworks, Natick, Mass.) circular statistics (Berens, 2009, Journal of Statistical Software 31:1-21) and image processing toolboxes were used to extract the quantitative metrics.

### Micro-Contact Printing

Traditional photolithographic techniques were utilized to prepare polydimethylsiloxane (PDMS, Sylgard 184, Dow Corning) stamps. In particular, masks bearing the desired square (50×50 um) features were designed in AUTOCAD (Autodesk, Inc.) and fabricated at the Harvard University Center for Nanoscale Systems (CNS, NNIN, Cambridge, Mass.). Using a mask-aligner (ABM Inc.) UV-light was shine through the custom-made mask into a silicon wafer (Wafer World) that had been spin-coated with SU-8 3005 photoresist (MicroChem Corp). The wafer was then developed in propylene glycol methyl ether acetate and utilized to cast PDMS stamps.

### Cell Culture Substrates

Polyacrylamide gels were engineered as previously described (McCain et al., 2012, Proc Natl Acad Sci USA 109:9881-9886). In particular to obtain a substrate stiffness of 13 kPa, the concentrations of streptavidin-acrylamide/bis were adjusted to a ratio of 7.5/0.3%. A 30 uL drop of polyacrylamide solution was added to a 25 mm activated coverslip and temporarily sandwiched with a 18 mm non-activated one. To transfer fibronectin islands, the thin hydrogel film was left to dry at 37° C. for 10 mins, sterilized with a UV-ozone cleaner (Jelight Company, Inc.) and then micro-contact printed using fibronectin cross-linked with biotin via Sulfo-NHS-LC-Biotin (Pierce).

### Primary Harvest

Ventricular myocytes were isolated from day 2 neonate Balb/c mice according to procedures approved by the Harvard University Animal Care and Use Committee. In brief, animals were sacrificed and ventricles removed and incubated in cold (4° C.) 0.1% (w/v) trypsin (USB Corp., Cleveland, Ohio) solution for approximately 12 hours. Ventricular tissue was further exposed to serial treatments (2 minutes each) of 0.1% (w/v) warm (37° C.) collagenase type II (Worthington Biochemical, Lakewood, N.J.) solution. Isolated neonate ventricular cardiac myocytes were seeded onto the engineered substrates at a density of 20,000 cells/cm² and maintained in culture medium consisting of Medium 199 (Invitrogen, Carlsbad, Calif.) supplemented with 10% (v/v) heat-inactivated fetal bovine serum (FBS), 10 mM HEPES, 20 mM glucose, 2 mM L-glutamine, 1.5 μL vitamin B-12, and 50 U/ml penicillin for the first 48 hours. After that, FBS concentration was switched to 2%.

### Stem Cell Culture

Human and murine induced pluripotent stem cells derived cardiomyocytes (hiCM and miCMs) were kindly provided by Cellular Dynamics Inc. (CDI, Madison, Wis.) and Axiogenesis (CorAt-iPS, Cologne, Germany). Cells were cultured in accord with manufacturers' recommendations. In particular, while hiCMs were seeded in 6-well plates in the presence of vendor-provided plating media, miCMs were enriched in T-25 flasks pre-coated with 10 mg/ml fibronectin (FN) (BD Biosciences, Bedford, Mass.) in the presence of manufacturer provided selection medium containing puromycin. After 72 hours, both cell types were dissociated with 0.25% trypsin-EDTA solution (Invitrogen, 25200-072) and re-seeded onto the engineered substrates at a density of 10,000 cells/cm².

### Image Preprocessing

Preprocessing steps were performed using the ImageJ-based FIJI platform (Schindelin et al., 2012, Nature Methods 9:676-682). In particular, the following plugins were utilized: i) the tubeness plugin was used to highlight the filamentous structure of sarcomeric α-actinin positive pixels (Sato et al., 1998, Medical Image Analysis 2: 143-168); ii) the OrientationJ plugin (Rezakhaniha et al., 2011, Biomech Model Mechanobiol 11:461-473) was used to calculate the orientations of each sarcomeric α-actinin positive pixels.

### The Sarcomere Packing Density

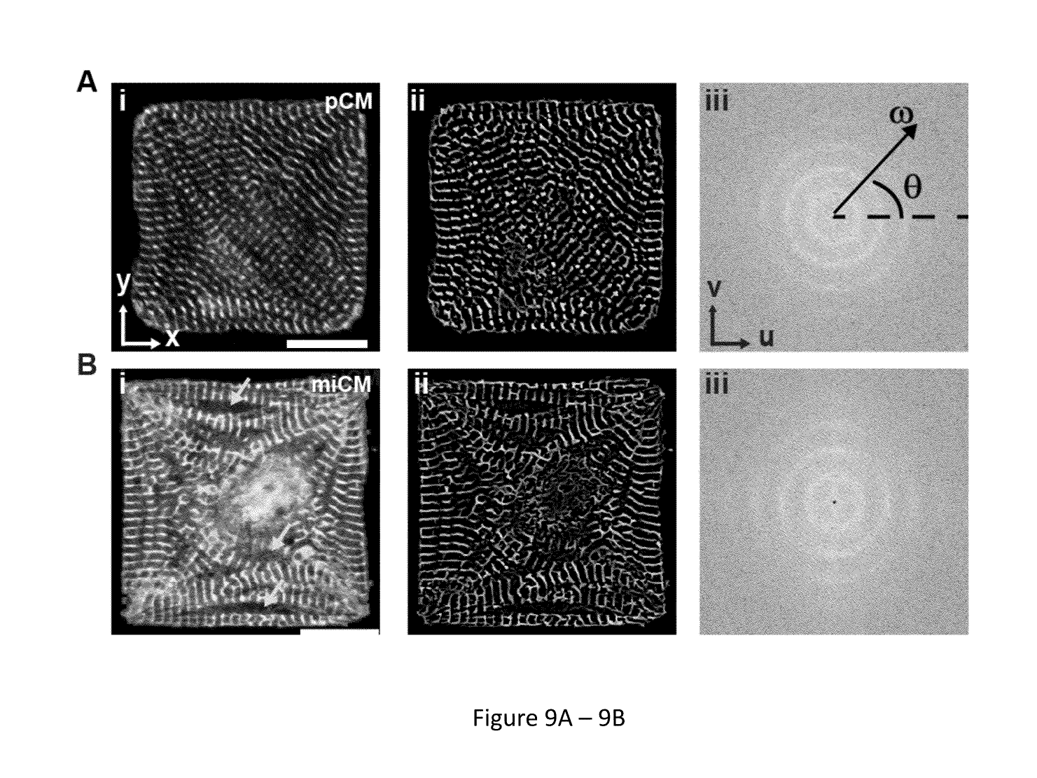

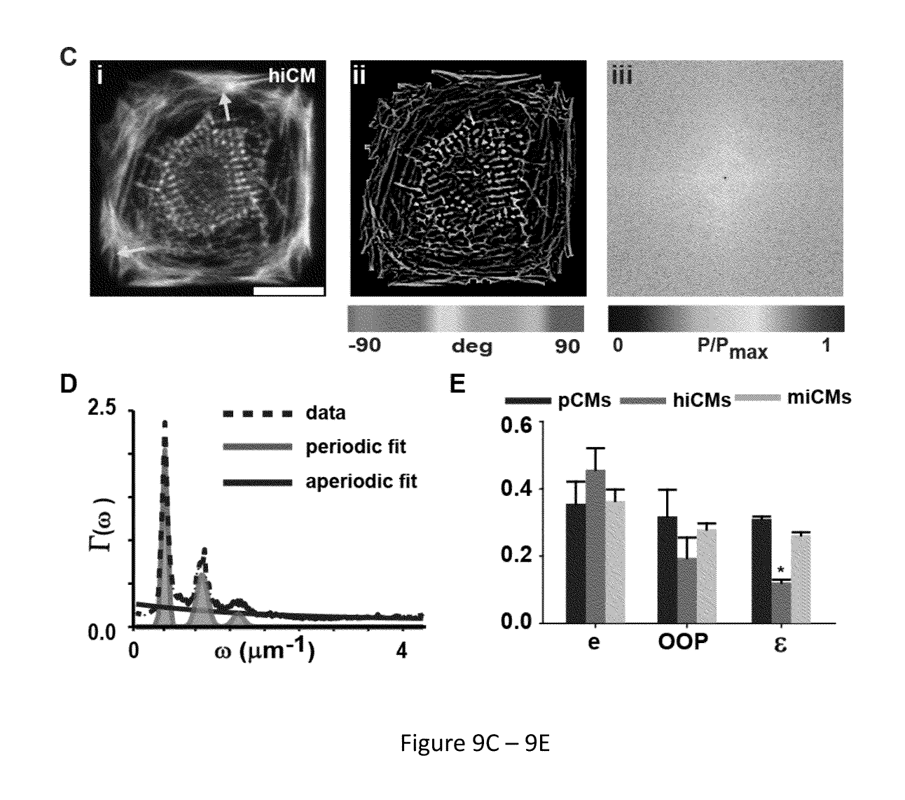

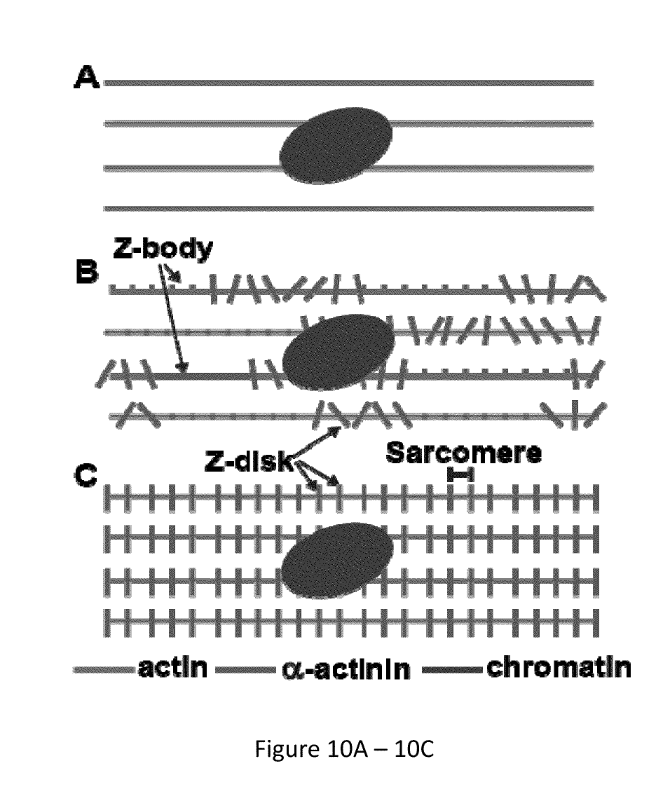

Force generation in striated muscle is associated with the vectorial summation of the contributions from all force generating units (Parker and Ingber, 2007, Philos Trans R Soc Lond B Biol Sci 362:1267-1279) known as sarcomeres. Sarcomeres are ˜2 μm long linear assemblies of cytoskeletal proteins whose concerted action generate a quantum of force parallel to the orientation of the sarcomere (McCain and Parker, 2011, Pflugers Arch 462:89-104). A common way to detect sarcomeres and their formation is via fluorescent immunolabeling of sarcomeric α-actinin (red in FIG. 9A(i)). This protein appears (Dabiri et al., 1997, Proc Natl Acad Sci USA 94:9493-9498; Parker et al., 2008, Circulation Research 103:340-342) to be diffuse in the cytosol during differentiation, then to assemble into puncta, known as Z-bodies (Sparrow and Schock, 2009, Nat Rev Mol Cell Biol 10:293-298), during the early phases of myofibrillogenesis and to localize to a regular lattice formed by Z-disks in mature myocytes (FIG. 10). The distance between two Z-disks is the sarcomere length. Here, a novel quantitative metric of cytoskeleton organization is presented, the sarcomere packing density, whose value increases as more sarcomeric α-actinin positive pixels are localized in periodically spaced Z-disks.

To calculate the sarcomeric packing density, the Fourier transform of the pre-processed sarcomere α-actinin micrograph K(x,y), was considered

$$F ( u , v ) = {\int \int }_{{ℜ}^{2}} K ( x , y ) {e}^{i 2 π ( x u + yv )} d x d y \qquad ( 5 )$$

and in particular its 2D power spectrum P(u, v)=|F(u, v)|² (where u and v are the coordinates of the Fourier domain and i indicates the complex unit). FIG. 9Aiii shows the power spectrum for the sarcomeric α-actinin micrograph in FIG. 9Ai. In this representation, each pixel corresponds to a planar wave traveling across the spatial domain with frequency and orientation given by the pixel polar coordinate (ω, θ) and power given by the pixel intensity P(u,v). By radially integrating the expression for the signal energy (E, eq 6) a 1D representation was obtained, Γ(ω) (blue traces in FIG. 10D), that exhibited periodic peaks modulated by a monotonically decreasing noise term.

$$E = {\int \int }_{{ℜ}^{2}} P ( u , v ) d u d v = {\int }_{0}^{∞} {\int }_{- π}^{π} P ( ω , θ ) ω d ω d θ = {\int }_{0}^{∞} [ ω ( {\int }_{- π}^{π} P ( ω , θ ) d θ ) ] d ω = {\int }_{0}^{∞} Γ ( ω ) d ω \qquad ( 6 )$$

To represent the periodic (\hat{Γ}ₚ, red curve in FIG. 9D) and aperiodic (\hat{Γ}ₐₚblack curve in FIG. 9D) components, the following expression was considered

$$\hat{Γ} ( ω ; γ ) = {\hat{Γ}}_{p} ( ω ; {γ}_{p} ) + {\hat{Γ}}_{ap} ( ω ; {γ}_{p} ) \{ \begin{matrix}{\hat{Γ}}_{ap} ( ω ; {γ}_{ap} ) = a {e}^{- ω / b} , & {γ}_{ap} = \{ a , b \} \\ {\hat{Γ}}_{p} ( ω ; {γ}_{p} ) = {\sum }_{k = 1}^{3} {a}_{k} {e}^{- {( ( ω - k {ω}_{0} ) / {b}_{k} )}^{2}} , & {γ}_{p} = {\{ {a}_{k} , {b}_{k} , {ω}_{0} \}}_{k = 1 , 2 , 3}\end{matrix} \qquad ( 7 )$$

By fitting the function \hat{Γ}(ω; γ) to the data Γ(ω) the values were determined for the set of parameters Γ={a, b, aₖ, bₖ, ω₉}_{k=1,2,3}. These parameters were utilized to determine the sarcomere length SL=ω₀⁻¹ and the sarcomere packing density (ε)

$$ɛ = {\int }_{D}^{ } {\hat{Γ}}_{p} ( ω ; {γ}_{p} ) d ω / {\int }_{ℜ}^{ } \hat{Γ} ( ω ; γ ) d ω \qquad ( 8 )$$

In particular the integration domain D at the numerator of eq 8 can be chosen so that only non-overlapping peaks are considered, further reducing the effect of artifacts and noise.

### Structural Phenotyping of Primary and Human Induced Pluripotent Stem Cell Derived Cardiomyocytes

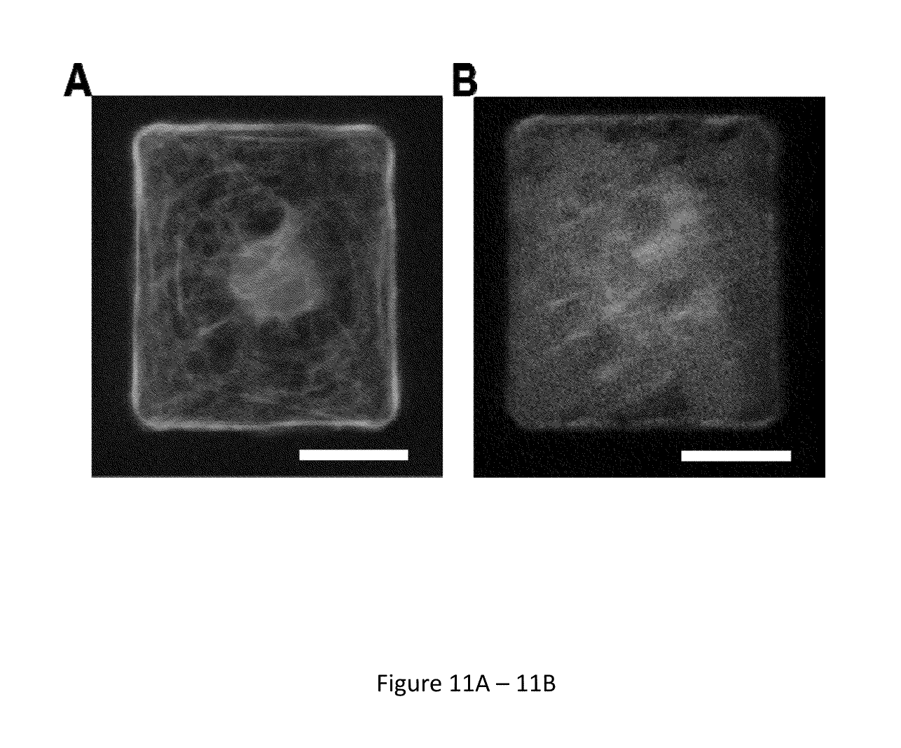

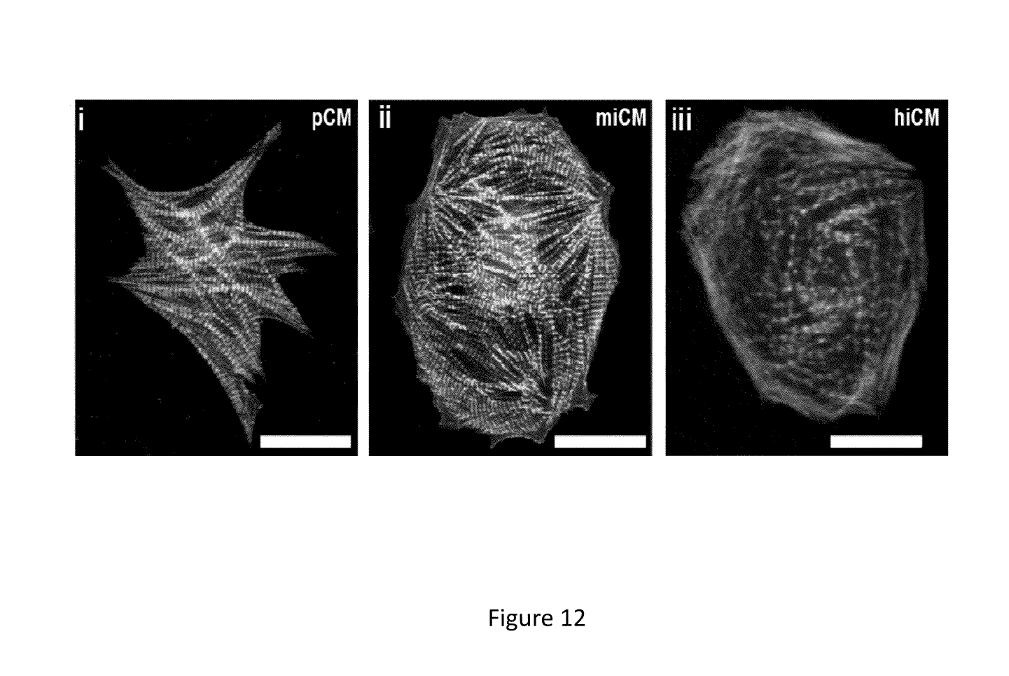

To showcase the ability of the sarcomere packing density to characterize the maturation of the cytoskeletal architecture in striated muscle, it was asked whether it could quantify the ability of human and murine induced pluripotent stem cells (respectively hiCMs and miCMs) to replicate the contractile cytoskeletal architecture observed in primary cells (pCMs). pCMs and iCMs were cultured on microcontact-printed hydrogels that mimic the native chemo-mechanical microenvironment and compared and contrasted their sarcomeric α-actinin organization. Qualitatively, the control pCMs showed mature cytoskeleton architecture (FIG. 9A(i)): the actin bundles (green) were uniformly distributed throughout the cytosol and displayed clear striations in correspondence of the Z-disks, where most of the α-actinin (red) signal localized; moreover, the cell nucleus (blue) was minimally deformed as expected for the particular cell geometry. Similarly, the cell cytoskeletal in miCMs was (FIG. 9B(i)) marked by striations of the actin bundles and regularly-arranged sarcomeric α-actinin positive Z-disks, although few regions displaying less dense packing of the myofibrils (white arrows) or Z-bodies were observed. In contrast, hiCMs exhibited actin and a-actin striations solely in the perinuclear region and arranged in ring-like myofibrils (red arrow in FIG. 9C(i)). Moreover, at the cell periphery, the actin and α-actinin signals were diffuse (white arrows) and resembled the cortical architecture (Lauffenburger and Horwitz, 1996, Cell 84:359-369) observed in migratory cells (FIG. 11). To quantify these differences, the experiment was restricted to 3 indicators: the nuclear eccentricity (e), an indicator adopted in existing structural phenotyping platforms; the orientational order parameter (OOP), that was previously utilized (Feinberg et al., 2012, Biomaterials 33:5732-5741) to estimate how similar the detected sarcomere orientations (FIGS. 9 Aii, Bii and Cii) are; and the Fourier transform based (FIGS. 9 Aiii, Biii and Ciii) sarcomere packing density (ε), that assesses the degree of development a contractile cytoskeleton. As shown in FIG. 9E, while pCMs and hiCMs showed similar nuclear morphology (respectively e=0.439±0.0812 and e=0.564±0.0796) and insignificantly different sarcomere alignment (respectively 00P=0.393±0.0980 and OOP=0.240±0.0749), pCMs did exhibit a significantly (p=0.001, n=3) higher density of well-formed sarcomeres (ξ=0.324±0.016) in relation to that of hiCMs (c=0.127±0.0217). Consistently with qualitative observations, while no significant differences between miCMs and pCMs or hiCMs were observed in the nuclear morphology (e=0.449±0.0422) and global sarcomere orientation (OOP=0.345±0.0224), the presence of periodically arranged sarcomeres (ε=0.262±0.0203) was significantly higher with respect to hiCMs (p=0.003, n=3) and similar to the level observed in pCMs (p=0.066, n=3).

Taken together these data suggest that pCMs and miCMs can be distinguished from hiCMs not only qualitatively, on the basis of structural hallmarks, such as cortical actin and ring-like myofibrils, but also quantitatively through a biophysically-sound metric, the sarcomere packing density, that permits a rigorous statistical classification.

Genetic, epigenetic and environmental factors all contribute to the pathophysiological state of cells and tissues. Recently, image processing and machine learning algorithms have been applied to correlate changes in cell morphology to underlying alterations of the genome (Crane, et al., 2012, Nature Methods 9: 977-980), expressome (Collinet et al., 2010, Nature 464:243-249) or proteome (Perlman et al., 2004, Science 306(5699):1194-1198) of the preparations. Here, the palette of morphometric features utilized in these studies has been extended, introducing a novel metric of cytoskeletal organization: the sarcomere packing density. As demonstrated herein, this metric can effectively distinguish the structural phenotypes of primary and stem cell derived cardiomyocytes using standard statistical tests. Notably, all myocytes considered in this study were positive for sarcomeric α-actinin suggesting that they would have been clustered in the same group based on the sole presence of this protein (Mummery et al., 2012, Circulation Research 111:344-358) or its transcript (Chin et al., 2009, Cell Stem Cell 5:111-123).

In previous methods, Fourier analysis has been adopted to estimate the sarcomere length. The automatic approach demonstrated herein offers significant advantages in that it considers the cytoskeleton within the entire cell, reducing the user-bias (Eliceiri et al., 2012, Nature Methods 9:697-710) introduced by manual selection of regions of interest in the spatial (Lundy et al., 2013, Stem Cells Dev 22(14):1991-2002) or Fourier (Wei et al., 2010, Circulation Research 107:520-531) domains. Moreover, the algorithm to calculate this metric not only yields a better estimate of the sarcomere length but also reveals the relative presence of well-formed sarcomeres. By normalizing the energy of the periodic component to the total energy of the sarcomeric α-actinin immunograph, a cytoskeletal signal-to-noise ratio can be estimated that is independent of the cell size and is bound by the interval [0, 1]; a desirable property for many machine-learning algorithms (Shamir et al., 2010, PLoS Comput Biol 6:e1000974).

In this study, metrics of cytoskeletal architecture were used to address the ability of human and murine induced pluripotent stem cell derived cardiomyocytes to assemble a contractile cytoskeleton similar to that observed in primary ventricular myocytes when subjected to engineered extracellular matrix guidance. When unconstrained, cells tend to assume a morphology dictated by their intrinsic cytoskeletal biases. For example, pCMs and miCMs tend to have pleomorphic shapes sustained by polarized cytoskeletal architectures, while hiCMs assumed ring-like cytoskeletal structures (FIG. 12). It was previously observed that, on centrally symmetric islands, primary cells could either respond to the ECM cues or retain their natural polarity (Grosberg et al., 2011, PLoS Comput Biol 7:e1001088) depending on the cell mechano-transduction ability (Sheehy et al., 2012, Biomech Model Mechanobiol. 11(8):1227-39). Based on these considerations, a square pattern was chosen, and it was observed that while the cytoskeletal architecture in pCMs and miCMs conformed to the provided boundary conditions, hiCMs retained the ring-like myofibril structure that typified their pleomorphic structural phenotype, suggesting that pathways regulating mechano-transduction (Sheehy et al., 2012) may be engaged differently in the immature hiCMs than in the mature pCMs and miCMs. This is consistent with the notion (Mummery et al., 2012, Circulation Research 111:344-358) that stem cells need to traverse a hierarchy of cardiac progenitor cells to become mature myocytes: in-vivo this process occurs over ˜260 days in human and ˜12 days in mouse (Sissman, 1970, Am J Cardiol 25:141-148). This suggests that longer time in culture may be beneficial in obtaining mature hiCMs, a fact further supported by a recent study (Lundy et al., 2013, Stem Cells Dev 22(14):1991-2002) where hiCMs cultured for longer than 100 days, showed strong evidence of structural and functional maturation.

Taken together, these considerations suggest that efforts for post-differentiation maturation strategies should be undertaken, to recapitulate, and possibly accelerate the natural maturation of stem cell derived cardiomyocytes in-vitro. In this context metrics of cytoskeletal architecture, integrated with traditional phenotyping methods (Beqqali et al., 2006, Stem Cells 24:1956-1967; He et al., 2003, Circulation Research 93:32-39), can enable quantitative characterization of the phenotype of iCMs at each development phase, and proves a valuable quality control tool for stem cell derived cardiomyocytes production (Fox, 2011, Nat Biotechnol 29:375-376).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for calculating a quality index of a differentiated cell, comprising:
measuring a differentiated cell by at least one metric;
calculating a normalized residue between the differentiated cell and a targeted cell; and
calculating a mean squared error (MSE) versus the target cell to define a value that represents the total difference between the differentiated cell and targeted cell based on the at least one measured metric.

2. The method of claim 1, wherein the at least one metric is selected from the group consisting of genetic information, electrophysiological information, structural information, and contractile information.

3. The method of claim 2, wherein the at least one metric comprises genetic information, electrophysiological information, structural information, and contractile information.

4. The method of claim 1, wherein the normalized residue is a strictly standardized mean difference (β).

5. The method of claim 4, wherein β is calculated according to the formula:
$$β = \frac{{μ}_{1} - {μ}_{2}}{\sqrt{{σ}_{1}^{2} + {σ}_{2}^{2}}}$$
where μ represents mean and σ represents standard deviation.

6. The method of claim 5, wherein MSE is calculated according to the formula:
$$MSE = \frac{1}{n} {\sum }_{i = 1}^{n} {β}_{i}^{2}$$

7. The method of claim 1, wherein the differentiated cell is derived from a potent cell.

8. The method of claim 7, wherein the potent cell is a stem cell.

9. The method of claim 8, wherein the differentiated cell is a myocyte.

10. The method of claim 9, wherein the at least one metric is a sarcomere packing density.

11. The method of claim 1, wherein information pertaining to the targeted cell is a predetermined value related to the at least one metric.

12. The method of claim 1, wherein a lower MSE value is indicative of greater similarity between the differentiated cell and the targeted cell.

13. A system for calculating a quality index of a differentiated cell, comprising a software platform run on a computing device that calculates a normalized residue between a differentiated cell and a targeted cell, and calculates a mean squared error (MSE) versus the target cell to define a value that represents the total difference between the differentiated cell and targeted cell based on at least one measured metric of the differentiated cell.

14. The system of claim 13, wherein the normalized residue is a strictly standardized mean difference (β).

15. The system of claim 14, wherein β is calculated according to the formula:
$$β = \frac{{μ}_{1} - {μ}_{2}}{\sqrt{{σ}_{1}^{2} + {σ}_{2}^{2}}}$$
where μ represents mean and σ represents standard deviation.

16. The system of claim 15, wherein MSE is calculated according to the formula:
$$MSE = \frac{1}{n} {\sum }_{i = 1}^{n} {β}_{i}^{2}$$

17. The system of claim 13, wherein the at least one metric is selected from the group consisting of genetic information, electrophysiological information, structural information, and contractile information.

18. The system of claim 17, wherein the at least one metric comprises genetic information, electrophysiological information, structural information, and contractile information.

19. The system of claim 13, wherein a lower MSE value is indicative of greater similarity between the differentiated cell and the targeted cell.

20. The system of claim 13, wherein the differentiated cell is derived from a potent cell.

21. The system of claim 19, wherein the potent cell is a stem cell.

22. The system of claim 13, wherein the differentiated cell is a myocyte.

23. The system of claim 21, wherein the at least one metric is a sarcomere packing density.
